SEQUENCE LISTING (66 sequences)

SEQ ID NO: 1 - DNA, 22 bp, Artificial Sequence
  Oligonucleotide Primer/Probe
tgctgtgtactacttcccaagg

SEQ ID NO: 2 - DNA, 20 bp, Artificial Sequence
  Oligonucleotide Primer/Probe
atggcaaacaagtggggata

SEQ ID NO: 3 - DNA, 14 bp, Artificial Sequence
  Oligonucleotide Primer/Probe
ccgagcgttctaaa

SEQ ID NO: 4 - DNA, 14 bp, Artificial Sequence
  Oligonucleotide Primer/Probe
ccgagcgttccaaa

SEQ ID NO: 5 - DNA, 20 bp, Artificial Sequence
  Oligonucleotide Primer/Probe
gatgggttcctttggagtca

SEQ ID NO: 6 - DNA, 20 bp, Artificial Sequence
  Oligonucleotide Primer/Probe
tcaacaatcatcccctcctc

SEQ ID NO: 7 - DNA, 15 bp, Artificial Sequence
  Oligonucleotide Primer/Probe
aagatatgagctgga

SEQ ID NO: 8 - DNA, 16 bp, Artificial Sequence
  Oligonucleotide Primer/Probe
atgagttggacataag

SEQ ID NO: 9 - DNA, 22 bp, Artificial Sequence
  Oligonucleotide Primer/Probe
aaaacatacccaatcaccatcc

SEQ ID NO: 10 - DNA, 22 bp, Artificial Sequence
  Oligonucleotide Primer/Probe
tttcagtggacaacacttctgg

SEQ ID NO: 11 - DNA, 15 bp, Artificial Sequence
  Oligonucleotide Primer/Probe
ccaggaaccaataca

SEQ ID NO: 12 - DNA, 16 bp, Artificial Sequence
  Oligonucleotide Primer/Probe
ccaggaaccattacat

SEQ ID NO: 13 - DNA, 20 bp, Artificial Sequence
  Oligonucleotide Primer/Probe
ttcctgaagagcggagacag

SEQ ID NO: 14 - DNA, 20 bp, Artificial Sequence
  Oligonucleotide Primer/Probe
gcacggagcttctcataagg

SEQ ID NO: 15 - DNA, 15 bp, Artificial Sequence
  Oligonucleotide Primer/Probe
cttccagagcagtgc

SEQ ID NO: 16 - DNA, 14 bp, Artificial Sequence
  Oligonucleotide Primer/Probe
ttccagagcagcgc

SEQ ID NO: 17 - DNA, 22 bp, Artificial Sequence
  Oligonucleotide Primer/Probe
gcctgatggtacaccggtcttg

SEQ ID NO: 18 - DNA, 24 bp, Artificial Sequence
  Oligonucleotide Primer/Probe
ttgggtctgttgcaccattaagaa

SEQ ID NO: 19 - DNA, 18 bp, Artificial Sequence
  Oligonucleotide Primer/Probe
ttcttgagatatcctcac

SEQ ID NO: 20 - DNA, 16 bp, Artificial Sequence
  Oligonucleotide Primer/Probe
ttcttgagacatcctc

SEQ ID NO: 21 - DNA, 20 bp, Artificial Sequence
  Oligonucleotide Primer/Probe
cgctaagcgtgtgctgttat

SEQ ID NO: 22 - DNA, 24 bp, Artificial Sequence
  Oligonucleotide Primer/Probe
tcacaggaatcaagttagacaggt

SEQ ID NO: 23 - DNA, 17 bp, Artificial Sequence
  Oligonucleotide Primer/Probe
ctaagtgcctagtctgt

SEQ ID NO: 24 - DNA, 17 bp, Artificial Sequence
  Oligonucleotide Primer/Probe
tcactaagtgcccagtc

SEQ ID NO: 25 - DNA, 22 bp, Artificial Sequence
  Oligonucleotide Primer/Probe
tcacaaaaacacacaacatcca

SEQ ID NO: 26 - DNA, 20 bp, Artificial Sequence
  Oligonucleotide Primer/Probe
accayatgcatgcaaatcca

SEQ ID NO: 27 - DNA, 18 bp, Artificial Sequence
  Oligonucleotide Primer/Probe
tcatacacatagatacac

SEQ ID NO: 28 - DNA, 18 bp, Artificial Sequence
  Oligonucleotide Primer/Probe
tcatacacatagacacac

SEQ ID NO: 29 - DNA, 22 bp, Artificial Sequence
  Oligonucleotide Primer/Probe
ggaacaatgtgtcttcaatgct

SEQ ID NO: 30 - DNA, 28 bp, Artificial Sequence
  Oligonucleotide Primer/Probe
aagaactagatcatactaactggcatgt

SEQ ID NO: 31 - DNA, 17 bp, Artificial Sequence
  Oligonucleotide Primer/Probe
cctaggaatcagtatgc

SEQ ID NO: 32 - DNA, 17 bp, Artificial Sequence
  Oligonucleotide Primer/Probe
cctaggaatcggtatgc

SEQ ID NO: 33 - DNA, 20 bp, Artificial Sequence
  Oligonucleotide Primer/Probe
ctccgttttccccatcttct

SEQ ID NO: 34 - DNA, 20 bp, Artificial Sequence
  Oligonucleotide Primer/Probe
agatctctcccgaaacacca

SEQ ID NO: 35 - DNA, 15 bp, Artificial Sequence
  Oligonucleotide Primer/Probe
tcgaatcccagctgt

SEQ ID NO: 36 - DNA, 14 bp, Artificial Sequence
  Oligonucleotide Primer/Probe
atcccggctgtctt

SEQ ID NO: 37 - DNA, 22 bp, Artificial Sequence
  Oligonucleotide Primer/Probe
tgctttttcctggctttctccc

SEQ ID NO: 38 - DNA, 25 bp, Artificial Sequence
  Oligonucleotide Primer/Probe
ccaccaggtcgacttggattagttt

SEQ ID NO: 39 - DNA, 15 bp, Artificial Sequence
  Oligonucleotide Primer/Probe
caccattccaaatgt

SEQ ID NO: 40 - DNA, 14 bp, Artificial Sequence
  Oligonucleotide Primer/Probe
accagtccaaatgt

SEQ ID NO: 41 - DNA, 20 bp, Artificial Sequence
  Oligonucleotide Primer/Probe
aggctaggagcaattggtga

SEQ ID NO: 42 - DNA, 24 bp, Artificial Sequence
  Oligonucleotide Primer/Probe
caaattggtaccataggcttcttc

SEQ ID NO: 43 - DNA, 15 bp, Artificial Sequence
  Oligonucleotide Primer/Probe
actctcactggttcc

SEQ ID NO: 44 - DNA, 15 bp, Artificial Sequence
  Oligonucleotide Primer/Probe
actctcagtggttcc

SEQ ID NO: 45 - DNA, 503 bp, Glycine max
atcaaagaataattaagcctctaatgcttaaaataactcaattcacgaacaaaaatgcttttgtttttcttttagtagattctactattgatttcttaacatgtttccatagggtaagtgttaatgaaaccctaataataataattctacaaagatgtaagaattaattattaagataaatggaacaataagaatcaagtagtgtcatcattcttgagtcaagttgccaacctgatgcattatccacatttgttcttctactaattagctccatacccaaggtcaatgagacattagcattcaccaaccgttcaatggaatgatcttgcttattggctcttgatacttcatcagtcgtggatgcatttatatgcactttaccatgctgtgtactacttcccaaggtttcattcttatgtgccgagcgttccaaatcaagatgattatcttctttaaccttattatccccacttgtttgccattgttcatggtcatagctgttt

SEQ ID NO: 46 - DNA, 99 bp, Glycine max
tgctgtgtactacttcccaaggtttcattcttatgtgccgagcgttccaaatcaagatgattatcttctttaaccttattatccccacttgtttgccat

SEQ ID NO: 47 - DNA, 481 bp, Glycine max
  misc_feature (59)..(60): n is a, c, g, or t
gcatgaagagtctagaccctcttgtccaatcaaatctcagaagactcatatacaaaccnntncagcaagagaagtcaaatatagatctttgagcaagcctccaaggcttagaagcagagggatttaggtgaaacagaggacgagagccctatgataatcctaagaaagcacagtaaagaaagaaatacatgttgtccagaagaaatagaagcttgatcaacaatcatcccctcctcctttccaaagaaaagaaagatatgagttggacataagtacatgactccaaaggaacccatcaagataccatcctccgatgactctacttcaccagacaatgaaataactccaactcttacttcacagccatctagtctaccactagtgcagtccaagaagtctctctctccaatcaacacatccaagtttccaatacttggttctacctagaagcggaaatggagaccacatggtcatagctgtt

SEQ ID NO: 48 - DNA, 81 bp, Glycine max
tcaacaatcatcccctcctcctttccaaagaaaagaaagatatgagttggacataagtacatgactccaaaggaacccatc

SEQ ID NO: 49 - DNA, 545 bp, Glycine max
  misc_feature (52)..(52): n is a, c, g, or t
tctaagttgtactaaccaacatcaatgtcaatagtgataacaaaaacatacncnnatattcaattttcactggttaaaatatcaaaattatctacaagaaattagggggaaataagagaaggaaggctctttaaatgaatttttgtttagatgacgaggaaccaaacttttcagtataaaaaaatagaaaaggaagtaagaccaaaaaaacatacccaatcaccatccggatcagcaccaggaaccaatacattgatctcgcttgactttgctgtagttatggatgcttctaaggagtctttactcagatatagctggcagccagaagtgttgtccactgaaattgtaggagctgcaccctgtgattagtaaaagcaagcatgagttcaacaaaatcagatttgacatatcaagtattcattgagtattaatattaaacttttatcctatgcatctcaccagttatatcattagtaagatcagtaaagctattaatatatttcttgggcagaataatatcatattcatgccaacagcattggaag

SEQ ID NO: 50 - DNA, 137 bp, Glycine max
aaaacatacccaatcaccatccggatcagcaccaggaaccaatacattgatctcgcttgactttgctgtagttatggatgcttctaaggagtctttactcagatatagctggcagccagaagtgttgtccactgaaa

SEQ ID NO: 51 - DNA, 464 bp, Glycine max
aatactaagctgaaggaatcgattgcaaaataataacccttagaaaacactacggctttgtaaatcataaaccctaagctctctcgcccctaatcctacgagctagctgaatgtccttgggcataatggtaaccctcttagcatgaatagcgcagaggttggtatcctcaaagagcccaacgaggtaggcctcagcggcttcctgaagagcggagacagcgctgctctggaagcgtagatcggtcttgaagtcctgagcgatttcccttacgagcctctggaaaggaagcttccttatgagaagctccgtgctcttctggtacttccgaatctccctcagagccactgtccccggcctgaaacggtggggcttcttcacgccgcccgtcgccggagcggacttgcgtgctgccttggtggcgagctgcttccttggagcttttcctccatggtcatagctgttt

SEQ ID NO: 52 - DNA, 113 bp, Glycine max
ttcctgaagagcggagacagcgctgctctggaagcgtagatcggtcttgaagtcctgagcgatttcccttacgagcctctggaaaggaagcttccttatgagaagctccgtgc

SEQ ID NO: 53 - DNA, 385 bp, Glycine max
caatgcatcctacaaaagctctagggcctgatggtacaccggtcttgttctataagcacttctgggaggtggttggtgaggatgtctcaagaattgtcgtagatattcttaatggtgcaacagacccaataggtattaatcaaactttcattgctctggtaactaaaattaagaatcctaactaggatggtgattttaggcctattaatctttgcaatgttgtttttaagattgttactaaaacaattgcaaatagattaaaaaaaaatgcttgggatagtgggggagagtcaaagtgcttttatgcctcatgtgcatattactgataatgctctcattgcttttgagtgtcttcattacacaaggacaaacatggtcatagctg

SEQ ID NO: 54 - DNA, 104 bp, Glycine max
gcctgatggtacaccggtcttgttctataagcacttctgggaggtggttggtgaggatgtctcaagaattgtcgtagatattcttaatggtgcaacagacccaa

SEQ ID NO: 55 - DNA, 479 bp, Glycine max
cggaactcatcatattctgtgacataaagagtgacgttcctctccagaaggatgttccgagagtgaacattctgggaatacctctcctaggtagcctcagaaataaatctggtagtgtcataaggctcctagggccgggaggcggtgcatttccttttccaggaggccatctgcatcaaaaaagaatcacaggaatcaagttagacaggtttttatctcaaactgaaaatagaaaaataaaacagaaaaacagactgggcacttagtgagactgactcgcttatgaaaataacagcacacgcttagcgcacagggcgcgcttagcacgacaacacaaaaacacaaactttggctgagcgagacacactcgcttagctgaaacaacataatggctaagcgagcatggctcaccaagccttattctctgacagagagctaattgcgcttagcgatactgactcacttagagtgacacacta

SEQ ID NO: 56 - DNA, 122 bp, Glycine max
tcacaggaatcaagttagacaggtttttatctcaaactgaaaatagaaaaataaaacagaaaaacagactgggcacttagtgagactgactcgcttatgaaaataacagcacacgcttagcg

SEQ ID NO: 57 - DNA, 528 bp, Glycine max
  misc_feature (56)..(60): n is a, c, g, or t
aaactaaaaataaaagacaataaacataagtctaagcttctcctccaacatcatcnnnnngaccttataaagtctcatctataagggtgacattcaacctaaacacaaacatgagataaacaaatggagagtgaggtatatctcacaaacatttaaaactcaaagaagacataaaaagaaaaaatatattataaataattgtatcacaaaaacacacaacatccacttcaaatgtctcatacacatagatacacatgcaagatgattcacatttaacttatggatttgcatgcatatggtaccattttgaaaatactttgtgaatgtagcctgggagtccatgtagtcgatagattgtcgcacaatggacaacttgacactatcactctcatcgagatgacactaccgacaatgcattagggtgttacggccttgtaaggatactccaacccatgttgttatgcgttggttagaggagaaaggaggaagaaggtgagagtaacggagagagacgcgttttccttgatt

SEQ ID NO: 58 - DNA, 97 bp, Glycine max
tcacaaaaacacacaacatccacttcaaatgtctcatacacatagatacacatgcaagatgattcacatttaacttatggatttgcatgcatatggt

SEQ ID NO: 59 - DNA, 487 bp, Glycine max
  misc_feature (475)..(475): n is a, c, g, or t
gatataaacaaagttatggcttgcaacgaaagtttctgaattacaggttgtagatttgaagtggaatagtgaaacataagggattttgagctacactagtatatacattcttcagtgacataggaacaatgtgtcttcaatgctacatttggcaaactaataggtgatagccaagttgatattttttttccctcctaggaatcggtatgcaaaatttatgaatagatctatagtctacatgccagttagtatgatctagttctttatattatccaatcataatcattttatattctcttagaaacatacctcgctgacatatgcttgactgttgtagattttctgttttgctatatttacatgaatggtgtatctcatattgccatggttgcttttgttgcttacccccaccccataaagaaaagtcccctacatacgatttctctaatggaaataagtttttttttggtaaganaangtggtgccc

SEQ ID NO: 60 - DNA, 142 bp, Glycine max
ggaacaatgtgtcttcaatgctacatttggcaaactaataggtgatagccaagttgatattttttttccctcctaggaatcggtatgcaaaatttatgaatagatctatagtctacatgccagttagtatgatctagttctt

SEQ ID NO: 61 - DNA, 417 bp, Glycine max
  misc_feature (407)..(410): n is a, c, g, or t
tatgaggaagcaccagaaccacttttattgctcaaggaatcagcagacccctttagggttaaacctcctaacttcttgatttcggccgtttccccagtattctgcttggatgcgttcgctgcagacttcaataaatccacaggagcagccagctccgttttccccatcttctgaaaagaagaccctgcgtctttcgtctcaggggttcgaatcccggctgtcttctgatggtgtttcgggagagatctagaactagttttcgagtccatcagaaatgctaaaaagtttcagttgagatcaaattgacctaatccacggcttagcatccatttaagtctccaaaaataacaaacaacctatcaaaggcagcaacatcacaaacaagtagtcagccatctccccatggnnnnancngtt

SEQ ID NO: 62 - DNA, 96 bp, Glycine max
ctccgttttccccatcttctgaaaagaagaccctgcgtctttcgtctcaggggttcgaatcccggctgtcttctgatggtgtttcgggagagatct

SEQ ID NO: 63 - DNA, 532 bp, Glycine max
aatagcctctgtgtaattcaattcataatgcgattcatctcattttagtatatgctttttcctggctttctccctttctccccaacaggggttacctcaaaccatattaactcgaggcacatttggaatggtgctccagaaactaatccaagtcgacctggtggggctaattttgctgccaaatgcaatatagtgttgttttcttgcacaatatatgataatataacatcctttgccgaccctatttcatgtatcagattgaagatggaggagtggcgatgcgaaaccgctgtgtgaattatactttgccttttgtcatccacttcccatatcaagctagggtgagaactaataagctctgacaagaatccaaaattaccaacttttgcagcattgaataatagtttagaaggttcacttatgattctaattgcccccgagaagttttgctggccaagaatacttttccaaagaaaattaaccaattcaaaaaactcatgctgtttctggcctgccatggtcatagcctgtt

SEQ ID NO: 64 - DNA, 112 bp, Glycine max
tgctttttcctggctttctccctttctccccaacaggggttacctcaaaccatattaactcgaggcacatttggaatggtgctccagaaactaatccaagtcgacctggtgg

SEQ ID NO: 65 - DNA, 393 bp, Glycine max
  misc_feature (279)..(280): n is a, c, g, or t
taatcctcttttactgcttcctaaaagatccattgcttccaactaagtgtccattctcacaatccaaatttgataattttctctgtaaaacacggtaacacggtaggaggctaggagcaattggtgaacaagtggcttctatgttcatggtgaccactctcagtggttccttaagaagaagcctatggtaccaatttgttgtttttgtaaagttgcaacagaaacatagacaactttgaaggaagaaacaggtttcttctttctttctttttttttttnnaanaaangnnangnnttttaanaannannnntttnaaaantnnggaaaaaaaaaaanntttnntnnnnnnnnaaaanaannntnnnannannnaaaacnnnnacccttnaaaa

SEQ ID NO: 66 - DNA, 91 bp, Glycine max
aggctaggagcaattggtgaacaagtggcttctatgttcatggtgaccactctcagtggttccttaagaagaagcctatggtaccaatttg

(19) United States
(12) Patent Application Publication
(10) Pub. No.: US 2013/0061347 A1
(43) Pub. Date: Mar. 7, 2013

(54) QTL ASSOCIATED WITH APHID RESISTANCE IN SOYBEANS AND METHODS OF THEIR USE

(71) Applicants: Julian M. Chaky, Urbandale, IA (US); Yanwen Xiong, Johnston, IA (US)

(72) Inventors: Julian M. Chaky, Urbandale, IA (US); Yanwen Xiong, Johnston, IA (US)

(21) Appl. No.: 13/606,556

(22) Filed: Sep. 7, 2012

Related U.S. Application Data
(60) Provisional application No. 61/531,665, filed on Sep. 7, 2011.

(51) Int. Cl. C12Q 1/68; A01H 1/02; C07H 21/04; A01H 5/10; C12N 5/04; G01N 33/53; A01H 5/00

(52) U.S. Cl. 800/265; 435/6.11; 800/312; 435/415; 536/24.3; 536/23.1

(57) ABSTRACT

This invention relates to methods of identifying and/or selecting soybean plants or germplasm that display improved antibiosis and/or antixenosis resistance to one or more biotypes of soybean aphid. In certain examples, the method comprises detecting at least one marker, haplotype, or marker profile that is associated with improved soybean aphid resistance. Also included are plants selected by the given methods and primers, probes, and kits useful for such methods.

### CROSS REFERENCE TO RELATED APPLICATIONS

This application claims priority to and benefit of U.S. Provisional Patent Application Ser. No. 61/531,665, filed Sep. 7, 2011, the specification of which is hereby incorporated by reference in its entirety.

### FIELD OF THE INVENTION

This invention relates to methods of identifying and/or selecting soybean plants or germplasm that display improved antibiosis and/or antixenosis resistance to one or more biotypes of soybean aphid.

### BACKGROUND

Soybeans (Glycine max L. Merr.) are a major cash crop and investment commodity in North America and elsewhere. Soybean oil is one of the most widely used edible oils, and soybeans are used worldwide both in animal feed and in human food production. Additionally, soybean utilization is expanding to industrial, manufacturing, and pharmaceutical applications. Soybeans are also vulnerable to more than one hundred different pathogens, with some pathogens having disastrous economic consequences. One important soybean pathogen is the soybean aphid, which can severely impact yield. Despite a large amount of effort expended in the art, commercial soybean crops are still largely susceptible to aphid infestation.

A native of Asia, the soybean aphid (Aphis glycines Matsumura) was first found in the Midwest in 2000 (Hartman, et al., “Occurrence and distribution of Aphis glycines on soybeans in Illinois in 2000 and its potential control,” (1 Feb. 2001), available at http://plantmanagementnetwork.org/phpldefault.asp). It rapidly spread throughout the region and into other parts of North America (Patterson and Ragsdale, “Assessing and managing risk from soybean aphids in the North Central States,” (11 Apr. 2002) available at http://planthealth.info/aphids_researchupdate.htm). High aphid populations can reduce crop production directly when their feeding causes severe damage such as stunting, leaf distortion, and reduced pod set (Sun, et al., (1990) Soybean Genet. News. 17:43-48). Yield losses attributed to the aphid in some fields in Minnesota during 2001, where several thousand aphids occurred on individual soybean plants, were >50% (Ostlie, K., “Managing soybean aphid,” (2 Oct. 2002), available at http://soybeans.umn.edu/crop/insects/aphid/aphid-publicationmanagingsba.htm), with an average loss of 101 to 202 kg/ha in those fields (Patterson and Ragsdale, “Assessing and managing risk from soybean aphids in the North Central States,” (11 Apr. 2002) available at http://planthealth.info/aphids_researchupdate.htm). In earlier reports from China, soybean yields were reduced up to 52% when there was an average of about 220 aphids per plant (Wang, et al. (1994) Plant Prot. (China) 20:12-13), and plantheight was decreased by about 210 mm after severe aphid infestation (Wang, et al. (1996) Soybean Sci. 15:243-247). An additional threat posed by the aphid is its ability to transmit certain plant viruses to soybean, such as Alfalfa mosaic virus, Soybean dwarf virus, and Soybean mosaic virus (Sama et al., “Varietal screening for resistance to the aphid, Aphis glycines, in soybean,” (1974) Research Reports 1968-1974, pp. 171-172; Iwaki et al., (1980) Plant Dis. 64:1027-1030; Hartman et al., “Occurrence and distribution of Aphis glycines on soybeans in Illinois in 2000 and its potential control,” (1 Feb. 2001), available at http://plantmanagementnetwork.org/phpldefault.asp; Hill et al. (1996) Appl. Entomol. 31:178-180; Clark and Perry (2002) Plant Dis. 86:1219-1222).

Currently, millions of dollars are spent annually on spraying insecticides to control soybean aphid infestation. An integral component of an integrated pest management (IPM) program to control aphids is plant resistance (Auclair, J. L., “Host plant resistance,” pp. 225-265 In P. Harrewijn (ed.) Aphids: Their biology, natural enemies, and control, Vol. C., Elsevier, N.Y. (1989); Harrewijn, P. and Minks, A. K., “Integrated aphid management: General aspects,” pp. 267-272, In A. K. Minks and P. Harrewijn (ed.) Aphids: Their biology, natural enemies, and control, Vol. C., Elsevier, N.Y. (1989)). Insect resistance can significantly reduce input costs for producers (Luginbill, J. P., “Developing resistant plants—The ideal method of controlling insects,” (1969) USDA, ARS. Prod. Res. Rep. 111, USGPO, Washington, D.C.).

There remains a need for soybean plants with improved resistance to soybean aphid and methods for identifying and selecting such plants.

### SUMMARY

This invention relates to methods of identifying and/or selecting soybean plants or germplasm that display improved antibiosis and/or antixenosis resistance to one or more biotypes of soybean aphid. In certain examples, the method comprises detecting at least one marker locus from outside of the Rag1, Rag2, and Rag3 intervals that is associated with improved soybean aphid resistance. In other examples, the method further comprises detecting one or more markers associated with one or more of Rag1, Rag2, or Rag3. In further examples, the method further comprises crossing a selected soybean plant with a second soybean plant. This invention further relates to markers, primers, probes, kits, systems, etc., useful for carrying out the methods described herein.

### BRIEF DESCRIPTION OF THE FIGURES

FIGS. 1A-1C, 2A-2C, and 3A-3D illustrate a genetic map comprising portions of the soybean genome comprising loci associated with aphid resistance. Map positions are provided in cM using a genetic map based upon Choi et al., “A Soybean Transcript Map: Gene Distribution, Haplotype and Single-Nucleotide Polymorphism Analysis” (2007) Genetics 176:685-96. FIGS. 1A-1C illustrate a genetic map of a portion of linkage group A2 comprising at least one locus associated with aphid resistance. FIGS. 2A-2C illustrate a genetic map of a portion of linkage group B1 comprising at least one locus associated with aphid resistance. FIGS. 3A-3D illustrate a genetic map of a portion of linkage group K comprising at least one locus associated with aphid resistance.

FIGS. 4A-4B provide a table of selected closely linked markers.

### SUMMARY OF THE SEQUENCES

SEQ ID NOs: 1-4 comprise nucleotide sequences of regions of the soybean genome, each capable of being used as a probe or primer, either alone or in combination, for the detection of marker locus S03517-1 on LG-A2. In certain examples, SEQ ID NOs: 1 and 2 are used as primers while SEQ ID NOs: 3-4 are used as probes.

SEQ ID NOs: 5-8 comprise nucleotide sequences of regions of the soybean genome, each capable of being used as a probe or primer, either alone or in combination, for the detection of marker locus S01629-1 on LG-A2. In certain examples, SEQ ID NOs: 5 and 6 are used as primers while SEQ ID NOs: 7 and 8 are used as probes.

SEQ ID NOs: 9-12 comprise nucleotide sequences of regions of the soybean genome, each capable of being used as a probe or primer, either alone or in combination, for the detection of marker locus S03253-1-A on LG-B1. In certain examples, SEQ ID NOs: 9 and 10 are used as primers while SEQ ID NOs: 11 and 12 are used as probes.

SEQ ID NOs: 13-16 comprise nucleotide sequences of regions of the soybean genome, each capable of being used as a probe or primer, either alone or in combination, for the detection of marker locus S01209-1-A on LG-B1. In certain examples, SEQ ID NOs: 13 and 14 are used as primers while SEQ ID NOs: 15 and 16 are used as probes.

SEQ ID NOs: 17-20 comprise nucleotide sequences of regions of the soybean genome, each capable of being used as a probe or primer, either alone or in combination, for the detection of marker locus S00737-1-A on LG-B1. In certain examples, SEQ ID NOs: 17 and 18 are used as primers while SEQ ID NOs: 19 and 20 are used as probes.

SEQ ID NOs: 21-24 comprise nucleotide sequences of regions of the soybean genome, each capable of being used as a probe or primer, either alone or in combination, for the detection of marker locus S01676-1 on LG-B1. In certain examples, SEQ ID NOs: 21 and 22 are used as primers while SEQ ID NOs: 23 and 24 are used as probes.

SEQ ID NOs: 25-28 comprise nucleotide sequences of regions of the soybean genome, each capable of being used as a probe or primer, either alone or in combination, for the detection of marker locus S01675-1 on LG-B1. In certain examples, SEQ ID NOs: 25 and 26 are used as primers while SEQ ID NOs: 27 and 28 are used as probes.

SEQ ID NOs: 29-32 comprise nucleotide sequences of regions of the soybean genome, each capable of being used as a probe or primer, either alone or in combination, for the detection of marker locus S04846-1-A on LG-K. In certain examples, SEQ ID NOs: 29 and 30 are used as primers while SEQ ID NOs: 31 and 32 are used as probes.

SEQ ID NOs: 33-36 comprise nucleotide sequences of regions of the soybean genome, each capable of being used as a probe or primer, either alone or in combination, for the detection of marker locus S04864-1-A on LG-K. In certain examples, SEQ ID NOs: 33 and 34 are used as primers while SEQ ID NOs: 35 and 36 are used as probes.

SEQ ID NOs: 37-40 comprise nucleotide sequences of regions of the soybean genome, each capable of being used as a probe or primer, either alone or in combination, for the detection of marker locus S00621-1 on LG-K. In certain examples, SEQ ID NOs: 37 and 38 are used as primers while SEQ ID NOs: 39 and 40 are used as probes.

SEQ ID NOs: 41-44 comprise nucleotide sequences of regions of the soybean genome, each capable of being used as a probe or primer, either alone or in combination, for the detection of marker locus S01781-1 on LG-K. In certain examples, SEQ ID NOs: 41 and 42 are used as primers while SEQ ID NOs: 43 and 44 are used as probes.

SEQ ID NO: 45 is the genomic DNA region encompassing marker locus S13517-1 on LG-A2.

SEQ ID NO: 46 is the amplicon produced by amplifying genomic DNA using SEQ ID NO: 1 as a forward or reverse primer in conjunction with SEQ ID NO: 2 as the other primer in the pair. This amplicon encompasses marker locus S13517-1 on LG-A2.

SEQ ID NO: 47 is the genomic DNA region encompassing marker locus S01629-1 on LG-A2.

SEQ ID NO: 48 is the amplicon produced by amplifying genomic DNA using SEQ ID NO: 5 as a forward or reverse primer in conjunction with SEQ ID NO: 6 as the other primer in the pair. This amplicon encompasses marker locus S01629-1 on LG-A2.

SEQ ID NO: 49 is the genomic DNA region encompassing marker locus S03253-1-A on LG-B1.

SEQ ID NO: 50 is the amplicon produced by amplifying genomic DNA using SEQ ID NO: 9 as a forward or reverse primer in conjunction with SEQ ID NO: 10 as the other primer in the pair. This amplicon encompasses marker locus S03253-1-A on LG-B1.

SEQ ID NO: 51 is the genomic DNA region encompassing marker locus S01209-1-A on LG-B1.

SEQ ID NO: 52 is the amplicon produced by amplifying genomic DNA using SEQ ID NO: 13 as a forward or reverse primer in conjunction with SEQ ID NO: 14 as the other primer in the pair. This amplicon encompasses marker locus S001209-1-A on LG-B1.

SEQ ID NO: 53 is the genomic DNA region encompassing marker locus 500737-1-A on LG-B1.

SEQ ID NO: 54 is the amplicon produced by amplifying genomic DNA using SEQ ID NO: 17 as a forward or reverse primer in conjunction with SEQ ID NO: 18 as the other primer in the pair. This amplicon encompasses marker locus S00737-1-A on LG-B1.

SEQ ID NO: 55 is the genomic DNA region encompassing marker locus S01676-1 on LG-B1.

SEQ ID NO: 56 is the amplicon produced by amplifying genomic DNA using SEQ ID NO: 21 as a forward or reverse primer in conjunction with SEQ ID NO: 22 as the other primer in the pair. This amplicon encompasses marker locus S01676-1 on LG-B1.

SEQ ID NO: 57 is the genomic DNA region encompassing marker locus S01675-1 on LG-B1.

SEQ ID NO: 58 is the amplicon produced by amplifying genomic DNA using SEQ ID NO: 25 as a forward or reverse primer in conjunction with SEQ ID NO: 26 as the other primer in the pair. This amplicon encompasses marker locus S01675-1 on LG-B1.

SEQ ID NO: 59 is the genomic DNA region encompassing marker locus 504846-1-A on LG-K.

SEQ ID NO: 60 is the amplicon produced by amplifying genomic DNA using SEQ ID NO: 29 as a forward or reverse primer in conjunction with SEQ ID NO: 30 as the other primer in the pair. This amplicon encompasses marker locus S04846-1-A on LG-K.

SEQ ID NO: 61 is the genomic DNA region encompassing marker locus S04864-1-A on LG-K.

SEQ ID NO: 62 is the amplicon produced by amplifying genomic DNA using SEQ ID NO: 33 as a forward or reverse primer in conjunction with SEQ ID NO: 34 as the other primer in the pair. This amplicon encompasses marker locus S04864-1-A on LG-K.

SEQ ID NO: 63 is the genomic DNA region encompassing marker locus S00621-1 on LG-K.

SEQ ID NO: 64 is the amplicon produced by amplifying genomic DNA using SEQ ID NO: 37 as a forward or reverse primer in conjunction with SEQ ID NO: 38 as the other primer in the pair. This amplicon encompasses marker locus 500621-1 on LG-K.

SEQ ID NO: 65 is the genomic DNA region encompassing marker locus S01781-1 on LG-K.

SEQ ID NO: 66 is the amplicon produced by amplifying genomic DNA using SEQ ID NO: 41 as a forward or reverse primer in conjunction with SEQ ID NO: 42 as the other primer in the pair. This amplicon encompasses marker locus S01781-1 on LG-K.

### DETAILED DESCRIPTION

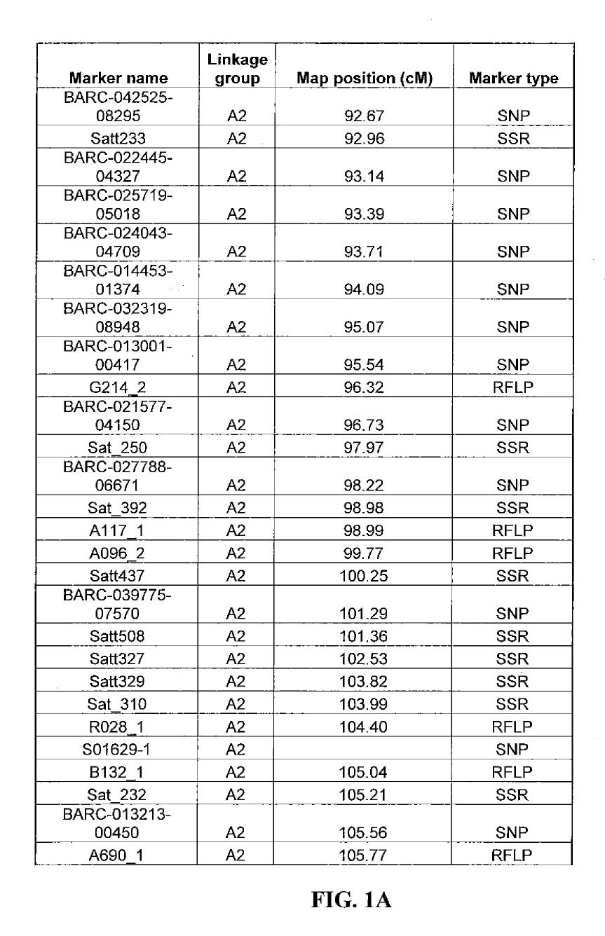

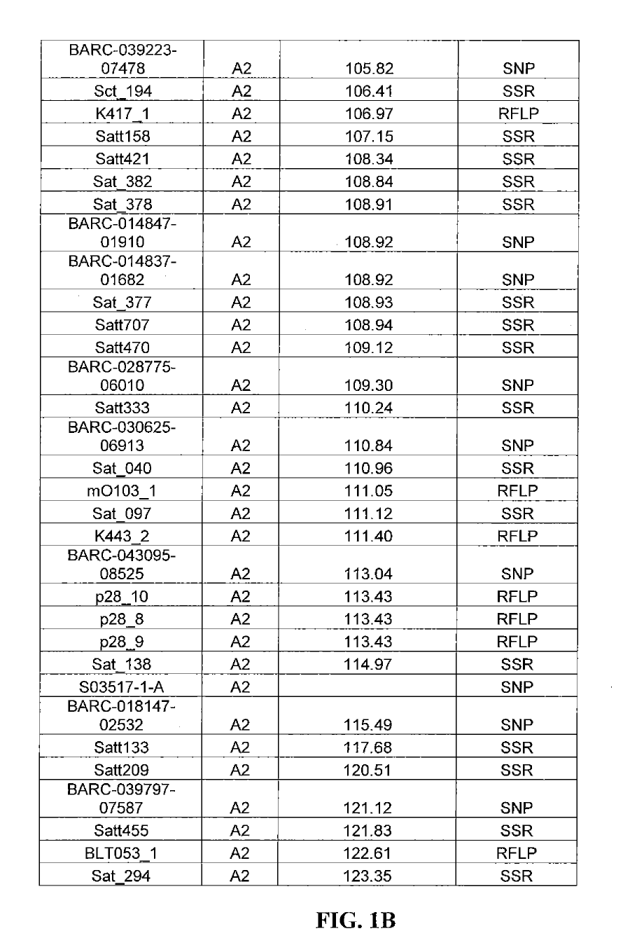

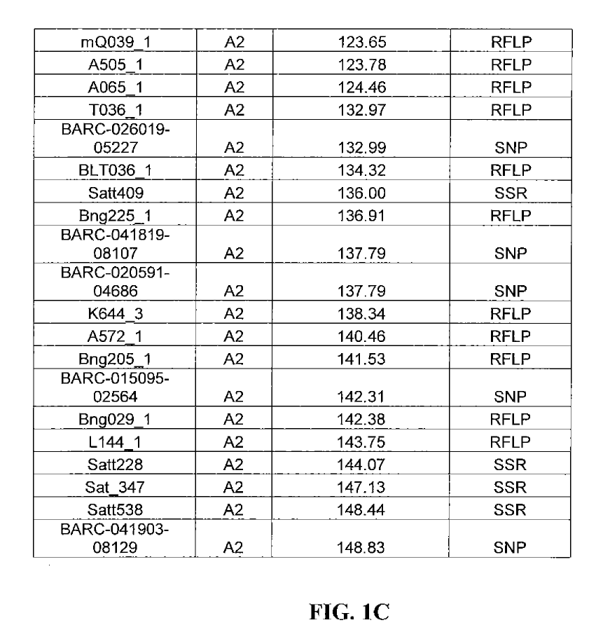

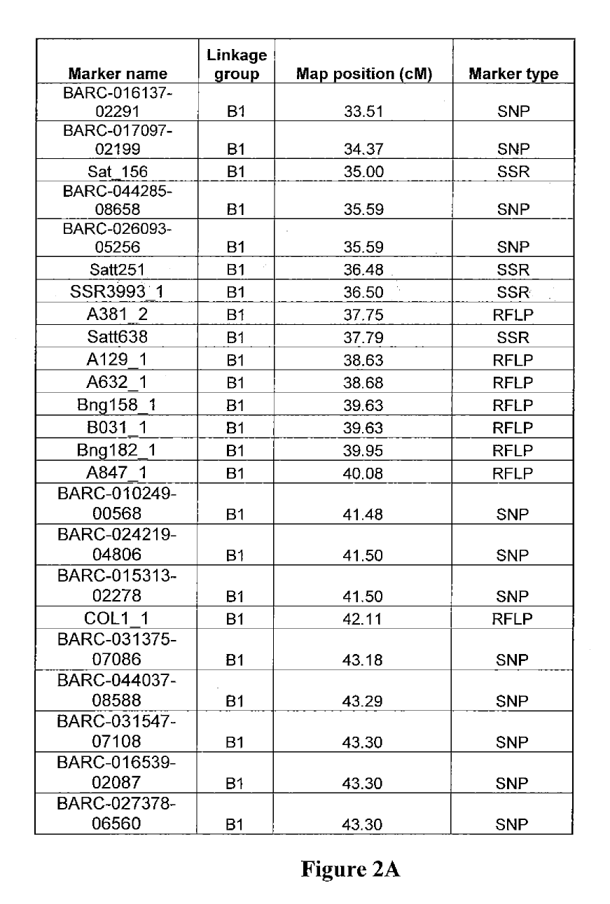

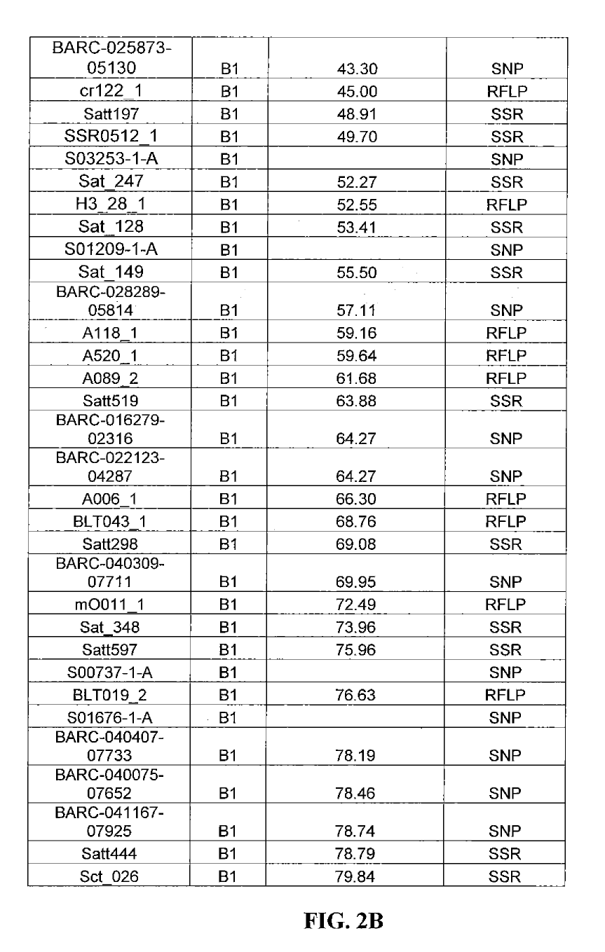

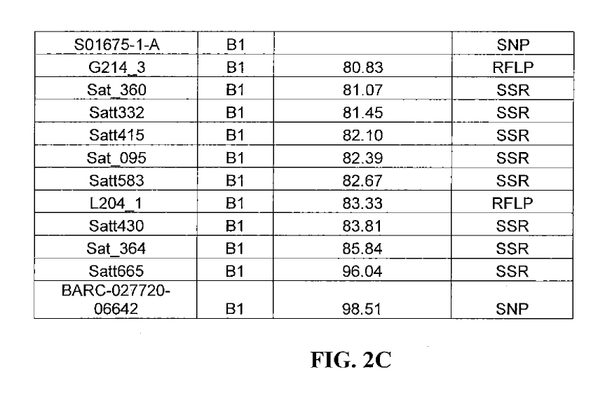

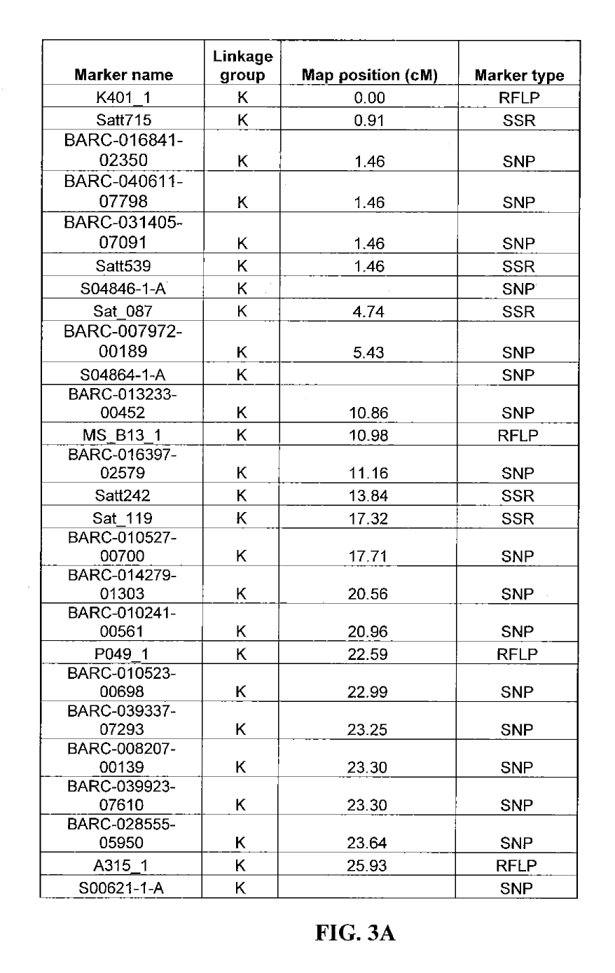

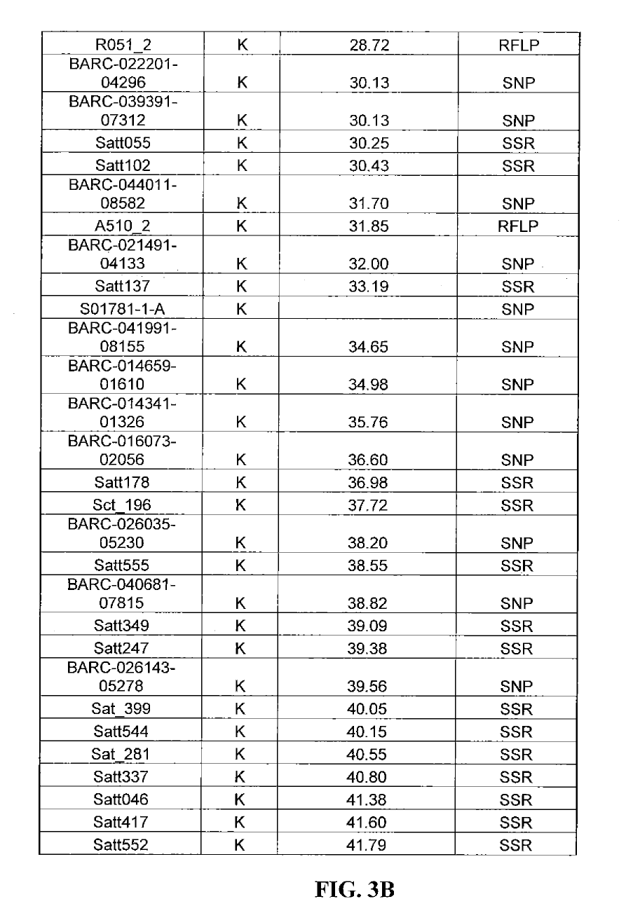

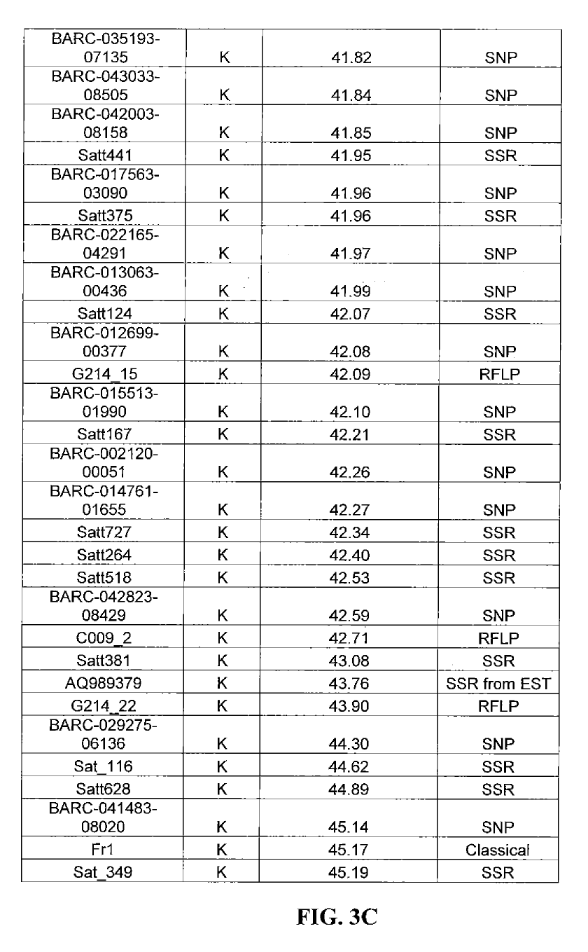

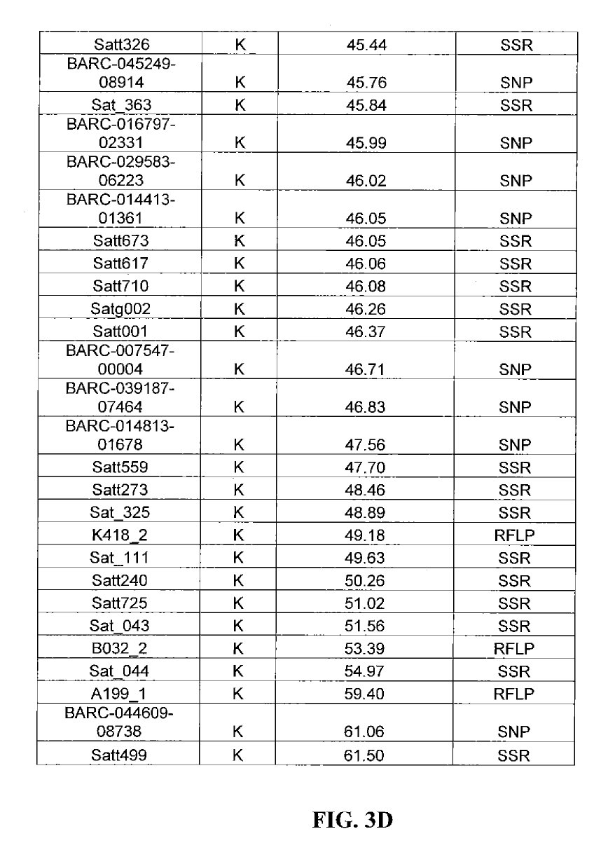

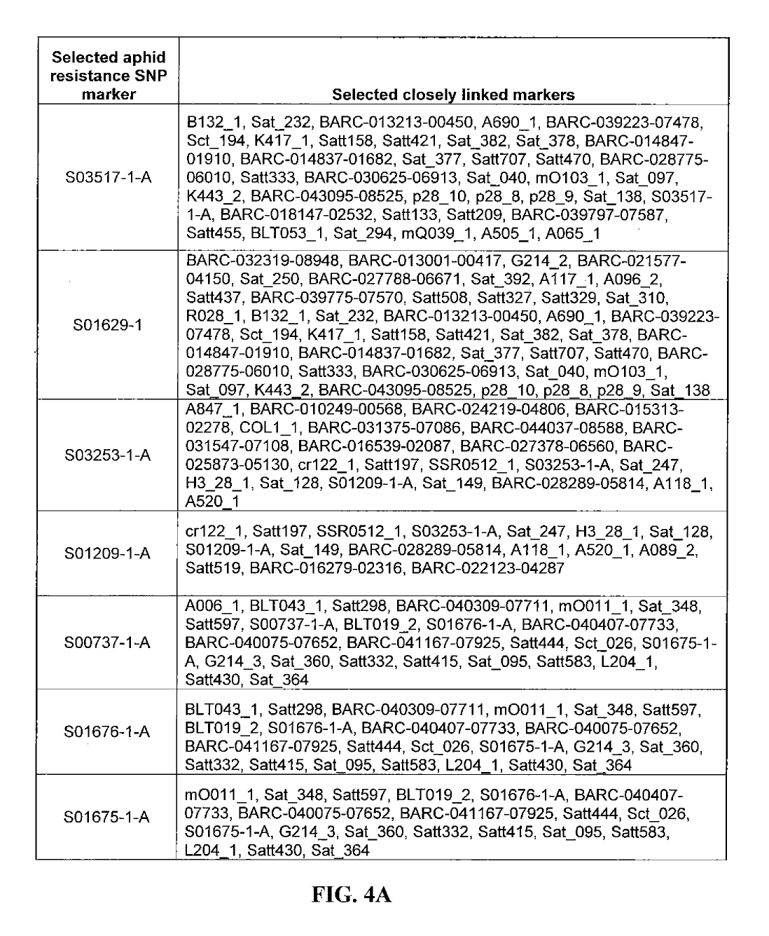

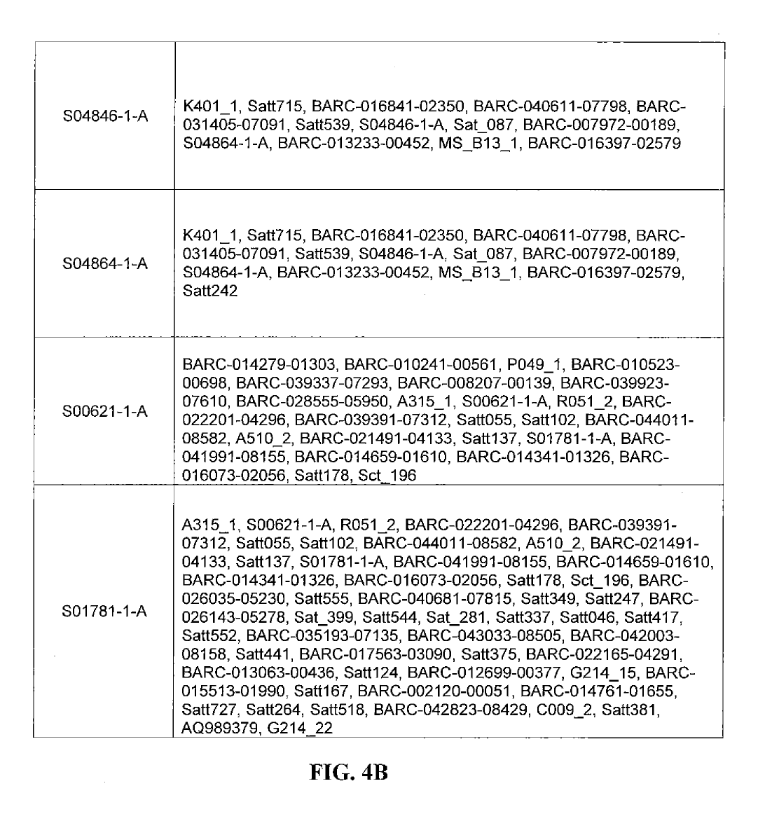

A novel method is provided for identifying a soybean plant or germplasm that displays improved resistance to one or more aphid biotypes, the method comprising detecting in the soybean plant or germplasm, or a part thereof, at least one marker that is associated with improved soybean aphid resistance, which marker locus is not found within the previously defined Rag1, Rag2, and Rag3 mapping intervals. In certain examples, the at least one marker is selected from the group consisting of 503517-1, S01629-1, S03253-1-A, S01209-1-A, S00737-1-A, S01676-1, $01675-1, S04846-1-A, S04864-1-A, S00621-1, S01781-1, and markers linked thereto. Examples of linked markers are provided in FIGS. 1A-1C, 2A-2C, and 3A-3D. In certain examples, such linked markers include markers within 50 cM of the identified SNP markers. In other examples, such linked markers include markers within 40 cM of the identified SNP markers. In still other examples, such linked markers include markers within 30 cM of the identified SNP markers. In further examples, such linked markers include markers within 20 cM of the identified SNP markers. In still further examples, such linked markers include markers within 10 cM of the identified SNP markers. In even further examples, such linked markers include markers within 5 cM of the identified SNP markers. Examples of closely linked markers are provided in FIGS. 4A-4B.

In other examples, the at least one marker is a marker capable of detecting a polymorphism at physical position selected from the group consisting of 40108281 on LG-A2/Chromosome 8, 25125032 on LG-A2/Chromosome 8, 8971115 on LG-B I/Chromosome 11, 9963410 on LG-B1/Chromosome 11, 17389892 on LG-B1/Chromosome 11, 18720097 on LG-B1/Chromosome 11, 18387725 on LG-B1/Chromosome 11, 887780 on LG-K/Chromosome 9, 1163103 on LG-K/Chromosome 9, 4700111 on LG-K/Chromosome 9, and 5021314 on LG-K/Chromosome 9, or a marker linked or closely linked thereto.

In other examples, the at least one marker comprises a marker localizing within a chromosome interval flanked by and including BARC-032319-08948 and A065_{—}1 on LG-A2, flanked by and including Sat_{—}250 and Sat_{—}294 on LG-A2, flanked by and including Satt437 and Satt209 on LG-A2, flanked by and including B132_{—}1 and A065_{—}1 on LG-A2, flanked by and including Sat_{—}232 and Sat_{—}294 on LG-A2, flanked by and including Satt333 and Satt209 on LG-A2, flanked by and including BARC-032319-08948 and Sat_{—}138 on LG-A2, flanked by and including Sat_{—}250 and Sat_{—}138 on LG-A2, flanked by and including Satt437 and Satt333 on LG-A2, flanked by and including A847_{—}1 and Sat_{—}364 on LG-B1, flanked by and including Satt197 and Satt430 on LG-B1, flanked by and including A847_{—}1 and BARC-022123-04287 on LG-B1, flanked by and including Satt197 and Satt519 on LG-B1, flanked by and including Satt197 and A520_{—}1 on LG-B1, flanked by and including A847_{—}1 and A520_{—}1 on LG-B1, flanked by and including Satt197 and Sat_{—}149 on LG-B1, flanked by and including Satt197 and Sat_{—}128 on LG-B1, flanked by and including cr122_{—}1 and BARC-022123-04287 on LG-B1, flanked by and including Satt197 and Satt519 on LG-B1, flanked by and including Sat_{—}247 and A520_{—}1 on LG-B1, flanked by and including A006_{—}1 and Sat_{—}364 on LG-B1, flanked by and including Sat_{—}348 and Satt430 on LG-B1, flanked by and including A006_{—}1 and Sat_{—}364 on LG-B1, flanked by and including Sat_{—}348 and Sat_{—}360 on LG-B1, flanked by and including Satt298 and Sat_{—}364 on LG-B1, flanked by and including Satt597 and Satt430 on LG-B1, flanked by and including K401_{—}1 and G214_{—}15 on LG-K, flanked by and including Satt715 and Satt124 on LG-K, flanked by and including K401_{—}1 and BARC-016397-02579 on LG-K, flanked by and including K401_{—}1 and BARC-007972-00189 on LG-K, flanked by and including K401_{—}1 and Sat_{—}087 on LG-K, flanked by and including K401_{—}1 and Satt242 on LG-K, flanked by and including BARC-014279-01303 and G214_{—}15 on LG-K, flanked by and including BARC-039337-07293 and Satt349 on LG-K, flanked by and including BARC-014279-01303 and Sct 196 on LG-K, flanked by and including BARC-039337-07293 and Satt137 on LG-K, flanked by and including A315_{—}1 and G215_{—}15 on LG-K, or flanked by and including Satt055 and Satt349 on LG-K. In yet further examples, the at least one marker comprises one or more markers within one or more of the genomic DNA regions of SEQ ID NOs: 45, 47, 49, 51, 53, 55, 57, 59, 61, 63, and 65. In other examples, the one or more marker locus detected comprises one or more markers within one or more of the amplicons of SEQ ID NOs: 46, 48, 50, 52, 54, 56, 58, 60, 62, 64, and 66. In certain examples, the method further comprises detecting one or more marker loci within one or more of the Rag1, Rag2, and Rag3 intervals.

In certain examples, a particular favorable is detected and/or selected. In some examples, the favorable allele is selected from the group consisting of allele C of S03517-1, allele T of S01629-1, allele T of S003253-1-A, allele G of S01209-1-A, allele G of S00737-1-A, allele A of S01676-1, allele T of S01675-1, allele G of S04846-1-A, allele G of S04864-1-A, allele C of S00621-1, and allele G of S01781-1. In other examples, the favorable allele is selected from the group consisting of allele C at physical position 40108281 on LG-A2, allele T at physical position 25125032 on LG-A2, allele T at physical position 8971115 on LG-B1, allele G at physical position 9963410 on LG-B1, allele G at physical position 17389892 on LG-B1, allele A at physical position 18720097 on LG-B1, allele T at physical position 18387725 on LG-B1, allele G at physical position 887780 on LG-K, allele G at physical position 1163103 on LG-K, allele C at physical position 4700111 on LG-K, and allele G at physical position 5021314 on LG-K. In other examples, a disfavored allele is detected and/or selected. In some examples, the disfavored allele is selected from the group consisting of allele T of S003517-1, allele C of S01629-1, allele A of S003253-1-A, allele of S01209-1-A, allele A of S00737-1-A, allele G of 801676-1, allele C of 801675-1, allele A of S004846-1-A, allele A of S04864-1-A, allele A of S00621-1, and allele C of S01781-1. In other examples, the disfavored allele is selected from the group consisting of allele T at physical position 40108281 on LG-A2, allele C at physical position 25125032 on LG-A2, allele A at physical position 9871115 on LG-B1, allele A at physical position 9963410 LG-B1, allele A at physical position 17389892 of LG-B1, allele G at physical position 18720097 on LG-B1, allele C at physical position 18387725 on LG-B1, allele A at physical position 887780 of LG-K, allele A at physical position 1163103 of LG-K, allele A at physical position 4700111 on LG-K, and allele C at physical position 5021314 on LG-K.

In other examples, the method comprises detecting and/or selecting plants with a certain aphid resistance haplotype on a particular linkage group, such as LG-A2, LG-B1 or LG-K. In still further examples, the method comprises detecting or selecting an aphid resistance marker profile comprising markers from multiple linkage groups, such as two or more of LG-A2, LG-B1, and LG-K. Examples of markers useful for generating such an aphid resistance haplotype or aphid resistance marker profile include S03517-1 on LG-A2, S01629-1 on LG-A2, S003253-1-A on LG-B1, S01209-1-A on LG-B1, 800737-1-A on LG-B1, S01676-1 on LG-B1, S01675-1 on LG-B1, S04846-1-A on LG-K, S04864-1-A on LG-K, 500621-1 on LG-K, and S01781-1 on LG-K, as well as markers capable of detecting a polymorphism at physical position selected from the group consisting of 40108281 on LG-A2/Chromosome 8, 25125032 on LG-A2/Chromosome 8, 8971115 on LG-B1/Chromosome 11, 9963410 on LG-B1/Chromosome 11, 17389892 on LG-B1/Chromosome 11, 18720097 on LG-B1/Chromosome 11, 18387725 on LG-B1/Chromosome 11, 887780 on LG-K/Chromosome 9, 1163103 on LG-K/Chromosome 9, 4700111 on LG-K/Chromosome 9, and 5021314 on LG-K/Chromosome 9.

In certain examples, the improved resistance comprises one or more of improved antibiosis resistance and improved antixenosis resistance. In other examples, the improved resistance comprises both improved antibiosis resistance and improved antixenosis resistance. In other examples, the improved soybean aphid resistance comprises improved resistance to at least two soybean aphid biotypes. In still other examples, the improved soybean aphid resistance comprises improved resistance to all three of soybean aphid biotypes 1, 2, and X.

In some examples, detecting comprises amplifying at least one of said marker loci or a portion thereof and detecting the resulting amplified marker amplicon. In certain examples, amplifying comprises: (a) admixing an amplification primer or amplification primer pair with a nucleic acid isolated from the first soybean plant or germplasm, wherein the primer or primer pair is complementary or partially complementary to at least a portion of the marker locus, and is capable of initiating DNA polymerization by a DNA polymerase using the soybean nucleic acid as a template; and, (b) extending the primer or primer pair in a DNA polymerization reaction comprising a DNA polymerase and a template nucleic acid to generate at least one amplicon. In certain embodiments, the amplification primer includes one or more of SEQ ID NOs: 1, 2, 5, 6, 9, 10, 13, 14, 17, 18, 21, 22, 25, 26, 29, 30, 33, 34, 37, 38, 41, and 42. In other examples, detecting further comprises providing a detectable probe. In certain embodiments, the detectable probe comprises one or more of SEQ ID NOs: 3, 4, 7, 8, 11, 12, 15, 16, 19, 20, 23, 24, 27, 28, 31, 32, 35, 36, 39, 40, 43, and 44. In further examples, the detectable probe comprises one or more of SEQ ID NOs: 46, 48, 50, 52, 54, 56, 58, 60, 62, 64, and 66, sequences complementary thereto, and portions thereof.

In still further examples, the information disclosed herein regarding markers, haplotypes, and marker profiles related to resistance to soybean aphid can be used to aid in the selection of breeding plants, lines, and populations containing improved resistance to soybean aphid for use in introgression of this trait into elite soybean germplasm, or germplasm of proven genetic superiority suitable for variety release. Also provided is a method for introgressing a soybean QTL, marker, haplotype, or marker profile associated with soybean aphid resistance into non-resistant soybean germplasm or less resistant soybean germplasm. According to the method, certain markers, haplotypes, and/or marker profiles are used to select soybean plants containing the improved resistance trait. Plants so selected can be used in a soybean breeding program. Through the process of introgression, the QTL, marker, haplotype, or marker profile associated with improved soybean aphid resistance is introduced from plants identified using marker-assisted selection (MAS) to other plants. According to the method, agronomically desirable plants and seeds can be produced containing the QTL, marker, haplotype, or marker profile associated with soybean aphid resistance from germplasm containing the QTL, marker, haplotype, or marker profile. Sources of improved soybean aphid resistance are disclosed below.

Also provided herein is a method for producing a soybean plant adapted for conferring improved soybean aphid resistance. First, donor soybean plants for a parental line containing the aphid resistance QTL, marker, haplotype, and/or marker profile are selected. According to the method, selection can be accomplished via MAS as explained herein. Selected plant material may represent, among others, an inbred line, a hybrid line, a heterogeneous population of soybean plants, or an individual plant. According to techniques well known in the art of plant breeding, this donor parental line is crossed with a second parental line. In some examples, the second parental line is a high yielding line. This cross produces a segregating plant population composed of genetically heterogeneous plants. Plants of the segregating plant population are screened for the soybean aphid resistance QTL, marker, haplotype, or marker profile. Further breeding may include, among other techniques, additional crosses with other lines, hybrids, backcrossing, or self-crossing. The result is a line of soybean plants that has improved resistance to soybean aphid and optionally also has other desirable traits from one or more other soybean lines.

Soybean plants, seeds, tissue cultures, variants, and mutants having improved soybean aphid resistance produced by the foregoing methods are also provided. Also provided are isolated nucleic acids, kits, and systems useful for the identification and selection methods disclosed herein.

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference herein for the purpose cited to the same extent as if each was specifically and individually indicated to be incorporated by reference herein.

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Additionally, as used herein, “comprising” is to be interpreted as specifying the presence of the stated features, integers, steps, or components as referred to, but does not preclude the presence or addition of one or more features, integers, steps, or components, or groups thereof. Thus, for example, a kit comprising one pair of oligonucleotide primers may have two or more pairs of oligonucleotide primers. Additionally, the term “comprising” is intended to include examples encompassed by the terms “consisting essentially of” and “consisting of:” Similarly, the term “consisting essentially of” is intended to include examples encompassed by the term “consisting of:”

Certain definitions used in the specification and claims are provided below. In order to provide a clear and consistent understanding of the specification and claims, including the scope to be given such terms, the following definitions are provided:

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant.

The term “amplifying” in the context of nucleic acid amplification is any process whereby additional copies of a selected nucleic acid (or a transcribed form thereof) are produced. An “amplicon” is an amplified nucleic acid, e.g., a nucleic acid that is produced by amplifying a template nucleic acid by any available amplification method.

“Backcrossing” is a process in which a breeder crosses a progeny variety back to one of the parental genotypes one or more times.

“Biotype” or “aphid biotype” means a subspecies of soybean aphid that share certain genetic traits or a specified genotype. There are currently three well-documented biotypes of soybean aphid: Urbana, Ill. (biotype 1), Wooster, Ohio (biotype 2), and Indiana (biotype 3). An additional biotype, referred to herein as biotype X, was collected from soybean fields in Lime Springs, Iowa.

The term “chromosome segment” designates a contiguous linear span of genomic DNA that resides in planta on a single chromosome. “Chromosome interval” refers to a chromosome segment defined by specific flanking marker loci.

“Cultivar” and “variety” are used synonymously and mean a group of plants within a species (e.g., Glycine max) that share certain genetic traits that separate them from other possible varieties within that species. Soybean cultivars are inbred lines produced after several generations of self-pollinations. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic linkage relationships among loci on one or more chromosomes (or linkage groups) within a given species, generally depicted in a diagrammatic or tabular form.

“Genotype” refers to the genetic constitution of a cell or organism.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Introgression” means the entry or introduction of a gene, QTL, marker, haplotype, marker profile, trait, or trait locus from the genome of one plant into the genome of another plant.

The terms “label” and “detectable label” refer to a molecule capable of detection. A detectable label can also include a combination of a reporter and a quencher, such as are employed in FRET probes or TaqMan™ probes. The term “reporter” refers to a substance or a portion thereof that is capable of exhibiting a detectable signal, which signal can be suppressed by a quencher. The detectable signal of the reporter is, e.g., fluorescence in the detectable range. The term “quencher” refers to a substance or portion thereof that is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a subline has been derived by inbreeding the seed from an individual soybean plant selected at the F3 to F5 generation until the residual segregating loci are “fixed” or homozygous across most or all loci. Commercial soybean varieties (or lines) are typically produced by aggregating (“bulking”) the self-pollinated progeny of a single F3 to F5 plant from a controlled cross between two genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to a phenomenon wherein alleles on the same chromosome tend to segregate together more often than expected by chance if their transmission was independent. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a map distance of 1.0 centiMorgan (1.0 cM).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosome segment are also genetically linked, typically within a genetic recombination distance of less than or equal to 50 centimorgans (cM), e.g., about 49, 40, 30, 20, 10, 9, 8, 7, 6, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 9%, 8%, 7%, 6%, 5%, 4%, 3%, 2%, 1%, 0.75%, 0.5%, or 0.25% or less. Closely linked markers display a cross over frequency with a given marker of about 10% or less (the given marker is within about 10 cM of a closely linked marker). Put another way, closely linked loci co-segregate at least about 90% of the time.

With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less.

When referring to the relationship between two genetic elements, such as a genetic element contributing to resistance and a proximal marker, “coupling” phase linkage indicates the state where the “favorable” allele at the resistance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for resistance) is physically linked with an “unfavorable” allele at the proximal marker locus, and the two “favorable” alleles are not inherited together (i.e., the two loci are “out of phase” with each other).

“Linkage disequilibrium” refers to a phenomenon wherein alleles tend to remain together in linkage groups when segregating from parents to offspring, with a greater frequency than expected from their individual frequencies.

“Linkage group” refers to traits or markers that generally co-segregate. A linkage group generally corresponds to a chromosomal region containing genetic material that encodes the traits or markers.

“Locus” is a defined segment of DNA.

A “map location,” “map position” or “relative map position” is an assigned location on a genetic map relative to linked genetic markers where a specified marker can be found within a given species. Map positions are generally provided in centimorgans. A “physical position” or “physical location” is the position, typically in nucleotide bases, of a particular nucleotide, such as a SNP nucleotide, on the chromosome.

“Mapping” is the process of defining the linkage relationships of loci through the use of genetic markers, populations segregating for the markers, and standard genetic principles of recombination frequency.

“Marker” or “molecular marker” is a term used to denote a nucleic acid or amino acid sequence that is sufficiently unique to characterize a specific locus on the genome. Any detectible polymorphic trait can be used as a marker so long as it is inherited differentially and exhibits linkage disequilibrium with a phenotypic trait of interest. A number of soybean markers have been mapped and linkage groups created, as described in Cregan, P. B., et al., “An Integrated Genetic Linkage Map of the Soybean Genome” (1999) Crop Science 39:1464-90, and more recently in Choi, et al., “A Soybean Transcript Map: Gene Distribution, Haplotype and Single-Nucleotide Polymorphism Analysis” (2007) Genetics 176:685-96. Many soybean markers are publicly available at the USDA affiliated soybase website (www.soybase.org). All markers are used to define a specific locus on the soybean genome. Large numbers of these markers have been mapped. Each marker is therefore an indicator of a specific segment of DNA, having a unique nucleotide sequence. The map positions provide a measure of the relative positions of particular markers with respect to one another. When a trait is stated to be linked to a given marker, it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed. Molecular markers have been widely used to determine genetic composition in soybeans. “Marker assisted selection” refers to the process of selecting a desired trait or traits in a plant or plants by detecting one or more nucleic acids from the plant, where the nucleic acid is linked to the desired trait, and then selecting the plant or germplasm possessing those one or more nucleic acids.

“Haplotype” refers to a combination of particular alleles present within a particular plant's genome at two or more linked marker loci, for instance at two or more loci on a particular linkage group. For instance, in one example, two specific marker loci on LG-B1 are used to define a haplotype for a particular plant. In another example, two specific marker loci on LG-K are used to define a haplotype for a particular plant. In still further examples, 3, 4, 5, 6, 7, 8, 9, 10, 11, 12, 13, 14, 15, 16, 17, 18, 19, 20, or more linked marker loci are used to define a haplotype for a particular plant.

As used herein, a “marker profile” means the combination of particular alleles present within a particular plant's genome at two or more marker loci that are not linked, for instance two or more loci on two or more different linkage groups. For instance, in one example, one marker locus on LG-A2 and one marker locus on LG-B1 are used to define a marker profile for a particular plant. In another example, one marker locus on LG-A2, one marker locus on LG-B1, and one marker locus on LG-K are used to define a marker profile for a particular plant. In certain other examples, a plant's marker profile comprises one or more haplotypes. For instance, in one example, one marker locus on LG-A2 and a haplotype made up of two marker loci on LG-B1 are used to define a marker profile for a particular plant. In another example, a haplotype made up of two marker loci on LG-B1 and a haplotype made up of two marker loci on LG-K are used to define a marker profile for a particular plant. In certain other examples, the marker profile further includes the Rag genes present in the particular. For instance, in one example, one marker locus on LG-A2 and the Rag genes present at one or more of the Rag1, Rag2, and Rag3 loci are used to define a marker profile for a particular plant. In another example, a haplotype made up of two marker loci on LG-B1, a haplotype made up of two marker loci on LG-K, and the Rag genes present at one or more of the Rag1, Rag2, and Rag3 loci are used to define a marker profile for a particular plant.

The term “plant” includes reference to an immature or mature whole plant, including a plant from which seed or grain or anthers have been removed. Seed or embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells, and the like.

“Polymorphism” means a change or difference between two related nucleic acids. A “nucleotide polymorphism” refers to a nucleotide that is different in one sequence when compared to a related sequence when the two nucleic acids are aligned for maximal correspondence.

“Polynucleotide,” “polynucleotide sequence,” “nucleic acid sequence,” “nucleic acid fragment,” and “oligonucleotide” are used interchangeably herein. These terms encompass nucleotide sequences and the like. A polynucleotide is a polymer of nucleotides that is single- or multi-stranded, that optionally contains synthetic, non-natural, or altered RNA or DNA nucleotide bases. A DNA polynucleotide may be comprised of one or more strands of cDNA, genomic DNA, synthetic DNA, or mixtures thereof.

“Primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are about 10 to nucleotides in length, but longer or shorter sequences can be employed. Primers may be provided in double-stranded form, though the single-stranded form is more typically used. A primer can further contain a detectable label, for example a 5′ end label.

“Probe” refers to an oligonucleotide (synthetic or occurring naturally) that is complementary (though not necessarily fully complementary) to a polynucleotide of interest and forms a duplexed structure by hybridization with at least one strand of the polynucleotide of interest. Typically, probes are oligonucleotides from 10 to 50 nucleotides in length, but longer or shorter sequences can be employed. A probe can further contain a detectable label.

“Quantitative trait loci” or “QTL” refer to the genetic elements controlling a quantitative trait.

“Rag genes,” “Rag intervals,” “Rag QTL,” and “Rag loci” refer to one or more of the Rag1, Rag2, and Rag3 genes and the chromosome segments or intervals on which they are located. Rag1 has been mapped to linkage group M in the vicinity of SSR markers Satt540 and Satt463 (Mian et al. (2008) Theor. Appl. Genet. 117:955-962; Kim et al. (2010) Theor. Appl. Genet. 120:1063-1071). In some examples, the Rag1 interval is defined as being flanked by and including markers Satt540 and BARC-016783-02329. In other examples, the Rag1 interval is defined as being flanked by and including markers BARC-039195-07466 and BARC-016783-02329. Rag2 has been mapped to linkage group F in the vicinity of SSR markers Satt334 and Sct_{—}033 (Mian et al. (2008) Theor. Appl. Genet. 117:955-962). In some examples, the Rag2 interval is defined as being flanked by and including markers Satt334 and Sat_{—}317. In other examples, the Rag2 interval is defined as being flanked by and including markers BARC-029823-06424 and Sct_{—}033. Rag3 is located on linkage group J in the vicinity of markers Sat_{—}339 and Satt414 (Zhang et al. (2010) Theor. Appl. Genet. 120:1183-1191). In some examples, the Rag 3 interval is defined as being flanked by and including markers Sat_{—}339 and Sct_{—}065. In other examples, the Rag3 interval is defined as being flanked by and including markers BARC-031195-07010 and Sat_{—}370.

“Recombination frequency” is the frequency of a crossing over event (recombination) between two genetic loci. Recombination frequency can be observed by following the segregation of markers and/or traits during meiosis.

“Resistance” and “improved resistance” are used interchangeably herein and refer to one or more of antibiosis resistance, antixenosis resistance, and tolerance to soybean aphid. “Antibiosis” refers to the plant's ability to reduce the survival, reproduction, and/or fecundity of the insect. “Antixenosis” refers to the plant's ability to deter the insect from feeding or identifying the plant as a food source. “Tolerance” refers to the plant's ability to withstand heavy infestation without significant yield loss. A “resistant plant” or “resistant plant variety” need not possess absolute or complete resistance to one or more soybean aphid biotypes. Instead, a “resistant plant,” “resistant plant variety,” or a plant or plant variety with “improved resistance” will have a level of resistance to at least one soybean aphid biotype that is higher than that of a comparable susceptible plant or variety.

“Self crossing” or “self pollination” or “selfing” is a process through which a breeder crosses a plant with itself; for example, a second generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome.

The term “yield” refers to the productivity per unit area of a particular plant product of commercial value. For example, yield of soybean is commonly measured in bushels of seed per acre or metric tons of seed per hectare per season. Yield is affected by both genetic and environmental factors. Yield is the final culmination of all agronomic traits.

Provided are markers, haplotypes, and marker profiles associated with improved resistance of soybeans to soybean aphid, as well as related primers and/or probes and methods for the use of any of the foregoing for identifying and/or selecting soybean plants with improved soybean aphid resistance. A method for determining the presence, or absence of at least one allele of a particular marker associated with soybean aphid resistance which is located outside of a Rag gene interval comprises analyzing genomic DNA from a soybean plant or germplasm to determine if at least one, or a plurality, of such markers is present or absent and if present, determining the allelic form of the marker(s). If a plurality of markers on a single linkage group is investigated, this information regarding the markers present in the particular plant or germplasm can be used to determine a haplotype for that plant/germplasm. If multiple markers or haplotypes on different linkage groups are deduced for a plant, a marker profile can in turn be assigned.

In certain examples, plants or germplasm are identified that have at least one favorable allele, marker, haplotype, and/or marker profile that positively correlates with resistance or improved resistance. However, in other examples, it is useful for exclusionary purposes during breeding to identify alleles, markers, haplotypes, or marker profiles that negatively correlate with resistance, for example to eliminate such plants or germplasm from subsequent rounds of breeding.

Any marker associated with a soybean aphid resistance QTL that is located outside of the Rag1, Rag2, and Rag3 intervals is useful. Any suitable type of marker can be used, including Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). Additionally, other types of molecular markers known in the art or phenotypic traits may also be used as markers in the methods.

Markers that map closer to a soybean aphid resistance QTL are generally preferred over markers that map farther from such a QTL. Marker loci are especially useful when they are closely linked to soybean aphid resistance QTL. Thus, in one example, marker loci display an inter-locus cross-over frequency of about 10% or less, about 9% or less, about 8% or less, about 7% or less, about 6% or less, about 5% or less, about 4% or less, about 3% or less, about 2% or less, about 1% or less, about 0.75% or less, about 0.5% or less, or about 0.25% or less with a soybean aphid resistance QTL to which they are linked. Thus, the loci are separated from the QTL to which they are linked by about 10 cM, 9 cM, 8 cM, 7 cM, 6 cM, 5 cM, 4 cM, 3 cM, 2 cM, 1 cM, 0.75 cM, 0.5 cM, or 0.25 cM or less.

In certain examples, multiple marker loci that collectively make up a haplotype or marker profile are investigated, for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, or more marker loci.

In addition to the markers discussed herein, information regarding useful soybean markers can be found, for example, on the USDA's Soybase website, available at www.soybase.org. One of skill in the art will recognize that the identification of favorable marker alleles may be germplasm-specific. One of skill will also recognize that methods for identifying the favorable alleles are routine and well known in the art, and furthermore, that the identification and use of such favorable alleles is well within the scope of the invention.

In some examples, marker profiles comprising two or more markers or haplotypes are provided. For instance, in one example, a particular marker or haplotype on LG-A2 and a particular marker or haplotype on LG-B1 define the marker profile of a particular plant. In another example, a particular marker or haplotype on LG-A2 and a particular marker or haplotype on LG-K define the marker profile of a particular plant. In a still further example, a particular marker or haplotype on LG-B1 and a particular marker or haplotype on LG-K define the marker profile of a particular plant. In an additional example, a particular marker or haplotype on LG-A2, a particular marker or haplotype on LG-B1, and a particular marker or haplotype on LG-K define the marker profile of a particular plant. In certain examples, the markers, haplotypes, or marker profiles comprise one or more of marker S03517-1 on LG-A2, S01629-1 on LG-A2, S03253-1-A on LG-B1, S01209-1-A on LG-B, S00737-1-A on LG-B, 501676-1 on LG-B1, S01675-1 on LG-B1, S04846-1-A on LG-K, S04864-1-A on LG-K, S00621-1 on LG-K, and S01781-1 on LG-K, or markers closely linked thereto, including the markers provided in FIGS. 1A-1C, 2A-2C, 3A-3D, and 4A-4B. In other examples, the marker profile further includes the Rag profile for that plant, e.g., the Rag1, Rag2, and/or Rag3 profile.

The use of marker assisted selection (MAS) to select a soybean plant or germplasm based upon detection of a particular marker, haplotype, or marker profile of interest is provided. For instance, in certain examples, a soybean plant or germplasm possessing a certain predetermined favorable marker allele or haplotype will be selected via MAS. In certain other examples, a soybean plant or germplasm possessing a certain predetermined favorable marker profile will be selected via MAS.

Using MAS, soybean plants or germplasm can be selected for markers or marker alleles that positively correlate with resistance, without actually raising soybean and measuring for resistance or improved resistance (or, contrawise, soybean plants can be selected against if they possess markers that negatively correlate with resistance or improved resistance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some examples, molecular markers are detected using a suitable amplification-based detection method. Typical amplification methods include various polymerase based replication methods, including the polymerase chain reaction (PCR), ligase mediated methods, such as the ligase chain reaction (LCR), and RNA polymerase based amplification (e.g., by transcription) methods. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage and Caruthers (1981) Tetrahedron Letts 22:1859-1862, e.g., using a commercially available automated synthesizer, e.g., as described in Needham-VanDevanter, et al. (1984) Nucleic Acids Res. 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair, or probe. For example, primers can be designed using any suitable software program, such as LASERGENE® or Primer3.

It is not intended that the primers be limited to generating an amplicon of any particular size. For example, the primers used to amplify the marker loci and alleles herein are not limited to amplifying the entire region of the relevant locus. In some examples, marker amplification produces an amplicon at least 20 nucleotides in length, or alternatively, at least 50 nucleotides in length, or alternatively, at least 100 nucleotides in length, or alternatively, at least 200 nucleotides in length, or alternatively, at least 300 nucleotides in length, or alternatively, at least 400 nucleotides in length, or alternatively, at least 500 nucleotides in length, or alternatively, at least 1000 nucleotides in length, or alternatively, at least 2000 nucleotides in length, or alternatively.

PCR, RT-PCR, and LCR are common amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous journal and patent references, such as Mullis, et al. (1987) U.S. Pat. No. 4,683,202; Arnmheim & Levinson (1990) C&EN 36-47; Kwoh et al. (1989) Proc. Natl. Acad. Sci. USA 86:1173; Guatelli et al. (1990) Proc. Natl. Acad. Sci. USA87:1874; Lomell et al. (1989) J. Clin. Chem 35:1826; Landegren et al. (1988) Science 241:1077-1080; Van Brunt (1990) Biotechnology 8:291-294; Wu and Wallace (1989) Gene 4:560; Barringer et al. (1990) Gene 89:117; and Sooknanan and Malek (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype alleles, such as SNP alleles, are provided. For example, exemplary primers and probes are provided in Table 1. However, one of skill will immediately recognize that other primer and probe sequences could also be used. For instance, primers to either side of the given primers can be used in place of the given primers, so long as the primers can amplify a region that includes the allele to be detected, as can primers and probes directed to other marker loci. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene, Oreg.); or Haugland (2001) Handbook of Fluorescent Probes and Research Chemicals Eighth Edition by Molecular Probes, Inc. (Eugene, Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland (2001) Handbook of Fluorescent Probes and Research Chemicals Eighth Edition by Molecular Probes, Inc. (Eugene, Oreg.), the content of which is incorporated herein by reference.

In certain examples, reporter-quencher pairs are selected from xanthene dyes including fluorescein and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters is the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from available from Applied Biosystems, and the like. Suitable examples of quenchers may be selected from 6-carboxy-tetramethyl-rhodamine, 4-(4-dimethylaminophenylazo)benzoic acid (DABYL), tetramethylrhodamine (TAMRA), BHQ-0™, BHQ-1™, BHQ-2™, and BHQ-3™, each of which are available from Biosearch Technologies, Inc. of Novato, Calif., QSY-7™, QSY-9™, QSY-21™ and QSY-35™, each of which are available from Molecular Probes, Inc., and the like.

In one aspect, real time PCR or LCR is performed on the amplification mixtures described herein, e.g., using molecular beacons or TaqMan™ probes. A molecular beacon (MB) is an oligonucleotide that, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al., (1995) Nucleic Acids Res. 26:2150-2155; Tyagi and Kramer (1996) Nature Biotechnology 14:303-308; Blok and Kramer (1997) Mol Cell Probes 11:187-194; Hsuih et al. (1997) J Clin Microbiol 34:501-507; Kostrikis et al. (1998) Science 279:1228-1229; Sokol et al. (1998) Proc. Natl. Acad. Sci. U.S.A. 95:11538-11543; Tyagi et al. (1998) Nature Biotechnology 16:49-53; Bonnet et al. (1999) Proc. Natl. Acad. Sci. U.S.A. 96:6171-6176; Fang et al. (1999) J. Am. Chem. Soc. 121:2921-2922; Marras et al. (1999) Genet. Anal. Biomol. Eng. 14:151-156; and, Vet et al. (1999) Proc. Natl. Acad. Sci. U.S.A. 96:6394-6399. Additional details regarding MB construction and use are also found in the patent literature, e.g., U.S. Pat. Nos. 5,925,517; 6,150,097; and 6,037,130.

Another real-time detection method is the 5′-exonuclease detection method, also called the TaqMan™ assay, as set forth in U.S. Pat. Nos. 5,804,375; 5,538,848; 5,487,972; and 5,210,015, each of which is hereby incorporated by reference in its entirety. In the TaqMan™ assay, a modified probe, typically 10-30 nucleotides in length, is employed during PCR that binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are typically attached to the probe within a few nucleotides of one another, usually within 30 nucleotides of one another, or within 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook et al. Molecular Cloning—A Laboratory Manual (3d ed.) Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”); and, PCR Protocols A Guide to Methods and Applications (Innis et alt, eds) Academic Press Inc. San Diego, Calif. (1990) (“Innis”). Additional details regarding detection of nucleic acids in plants can also be found, e.g., in Plant Molecular Biology (1993) Croy (ed.) BIOS Scientific Publishers, Inc.

Other techniques for detecting SNPs can also be employed, such as allele specific hybridization (ASH) or nucleic acid sequencing techniques. ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 13-18 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Introgression of soybean aphid resistance into non-resistant or less-resistant soybean germplasm is provided. Any method for introgressing a QTL or marker into soybean plants known to one of skill in the art can be used. Typically, a first soybean germplasm that contains resistance to soybean aphid derived from a particular marker, haplotype, or marker profile and a second soybean germplasm that lacks such resistance derived from the marker, haplotype, or marker profile are provided. The first soybean germplasm may be crossed with the second soybean germplasm to provide progeny soybean germplasm. These progeny germplasm are screened to determine the presence of soybean aphid resistance derived from the marker, haplotype, or marker profile, and progeny that tests positive for the presence of resistance derived from the marker, haplotype, or marker profile are selected as being soybean germplasm into which the marker, haplotype, or marker profile has been introgressed. Methods for performing such screening are well known in the art and any suitable method can be used.

One application of MAS is to use the resistance or improved resistance markers, haplotypes, or marker profiles to increase the efficiency of an introgression or backcrossing effort aimed at introducing a resistance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (resistance, along with any other available markers for yield, disease resistance, etc.). Any of the disclosed marker alleles, haplotypes, or marker profiles can be introduced into a soybean line via introgression, by traditional breeding (or introduced via transformation, or both) to yield a soybean plant with superior agronomic performance. The number of alleles associated with resistance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with resistance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant that comprises at least one of the markers, haplotypes, or marker profiles associated with resistance, such that the progeny are capable of inheriting the marker, haplotype, or marker profile.

Often, a method is applied to at least one related soybean plant such as from progenitor or descendant lines in the subject soybean plants pedigree such that inheritance of the desired resistance can be traced. The number of generations separating the soybean plants being subject to the methods will generally be from 1 to 20, commonly 1 to 5, and typically 1, 2, or 3 generations of separation, and quite often a direct descendant or parent of the soybean plant will be subject to the method (i.e., 1 generation of separation).

Genetic diversity is important for long-term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool.

For example, the markers, haplotypes, primers, probes, and marker profiles can be used for MAS involving crosses of elite lines to exotic soybean lines (elite X exotic) by subjecting the segregating progeny to MAS to maintain major yield alleles, along with the resistance marker alleles herein.

As an alternative to standard breeding methods of introducing traits of interest into soybean (e.g., introgression), transgenic approaches can also be used to create transgenic plants with the desired traits. In these methods, exogenous nucleic acids that encode a desired QTL, marker, haplotype, or marker profile are introduced into target plants or germplasm. For example, a nucleic acid that codes for a resistance trait is cloned, e.g., via positional cloning, and introduced into a target plant or germplasm.

Three types of soybean aphid resistance have been described: antibiosis, antixenosis, and tolerance. Experienced plant breeders can recognize resistant soybean plants in the field, and can select the resistant individuals or populations for breeding purposes or for propagation. In this context, the plant breeder recognizes “resistant” and “non-resistant” or “susceptible” soybean plants. However, plant resistance is a phenotypic spectrum consisting of extremes in resistance and susceptibility, as well as a continuum of intermediate resistance phenotypes. Evaluation of these intermediate phenotypes using reproducible assays are of value to scientists who seek to identify genetic loci that impart resistance, to conduct marker assisted selection for resistance populations, and to use introgression techniques to breed a resistance trait into an elite soybean line, for example.

To that end, screening and selection of resistant soybean plants may be performed, for example, by exposing plants to soybean aphid in a live aphid assay and selecting those plants showing resistance to aphids. Such assays can be used to test for each type of soybean aphid resistance, and may be any such assay known to the art, e.g., as described in Hill et al. (2004) Crop Science 44:98-106, Hill et al. (2004) J. Economic Entomology 97:1071-1077, or Li et al. (2004) J. Economic Entomology 97:1106-1111, each of which is incorporated herein by reference in its entirety, or as described in the Examples hereof.

In some examples, a kit or an automated system for detecting markers, haplotypes, and marker profiles and/or correlating the markers, haplotypes, and marker profiles with a desired phenotype (e.g., resistance) are provided. Thus, a typical kit can include a set of marker probes and/or primers configured to detect at least one favorable allele of one or more marker locus associated with resistance or improved resistance to a soybean aphid infestation. These probes or primers can be configured, for example, to detect the marker alleles noted in the tables and examples herein, e.g., using any available allele detection format, such as solid or liquid phase array based detection, microfluidic-based sample detection, etc. The kits can further include packaging materials for packaging the probes, primers, or instructions; controls, such as control amplification reactions that include probes, primers, and/or template nucleic acids for amplifications; molecular size markers; or the like.

A typical system can also include a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, scanning detectors, phototubes and photodiodes, microscope stations, galvo-scans, microfluidic nucleic acid amplification detection appliances, and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used. Typical detector examples include light (e.g., fluorescence) detectors or radioactivity detectors. For example, detection of a light emission (e.g., a fluorescence emission) or other probe label is indicative of the presence or absence of a marker allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele. The detector(s) optionally monitors one or a plurality of signals from an amplification reaction. For example, the detector can monitor optical signals that correspond to “real time” amplification assay results.

System or kit instructions that describe how to use the system or kit or that correlate the presence or absence of the favorable allele with the predicted resistance are also provided. For example, the instructions can include at least one look-up table that includes a correlation between the presence or absence of the favorable alleles or SNP profiles and the predicted resistance or improved resistance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector. As noted, in one typical example, the system instructions include at least one look-up table that includes a correlation between the presence or absence of the favorable alleles and predicted resistance or improved resistance. The instructions also typically include instructions providing a user interface with the system, e.g., to permit a user to view results of a sample analysis and to input parameters into the system.

Isolated nucleic acids comprising a nucleic acid sequence coding for resistance to soybean aphid, or sequences complementary thereto, are also included. In certain examples, the isolated nucleic acids are capable of hybridizing under stringent conditions to nucleic acids of a soybean cultivar resistant to soybean, for instance to particular markers, including one or more of S03517-1, S01629-1, S03253-1-A, S01209-1-A, S00737-1-A, S01676-1, S01675-1, S04846-1-A, S04864-1-A, S00621-1, and S01781-1. Vectors comprising such nucleic acids, expression products of such vectors expressed in a host compatible therewith, antibodies to the expression product (both polyclonal and monoclonal), and antisense nucleic acids are also included.

As the parental line having soybean aphid resistance, any line known to the art or disclosed herein may be used. Also included are soybean plants produced by any of the foregoing methods. Seed of a soybean germplasm produced by crossing a soybean variety having a marker, haplotype, or marker profile associated with soybean aphid resistance with a soybean variety lacking such marker, haplotype, or marker profile, and progeny thereof, is also included.

The present invention is illustrated by the following examples. The foregoing and following description of the present invention and the various examples are not intended to be limiting of the invention but rather are illustrative thereof. Hence, it will be understood that the invention is not limited to the specific details of these examples.

### EXAMPLES

### Example 1

### Phenotyping a Mapping Population

### Aphid Isolates:

The three soybean aphid biotype colonies are maintained in a growth chamber at the Dallas Center Containment Facility (Dallas Center, Iowa). The colonies are maintained on a continuous supply of Pioneer soybean variety 90M60. Two colonies of Urbana, Ill. (biotype 1) and Wooster, Ohio (biotype 2) were obtained from Brian Diers at the University of Illinois. An additional soybean aphid biotype (herein referred to as biotype X) was collected from soybean fields in Lime Springs, Iowa. The colonies are maintained in isolated tents to avoid mixing.

### Summer Field Trial Experiment:

A field experiment was conducted in 2009 at four locations across the Midwest to evaluate the resistance of two Rag1 donors, Dowling and Pioneer variety 95B97. The plots were planted in short rows, with 28 resistant lines and 6 susceptible checks arranged in random order. The plots were scored when the fields became naturally infested with soybean aphids over the growing season. The plants were visually phenotyped when the susceptible check Pioneer variety 93B15 was covered with soybean aphids. The 95B97 lines scored a 9 compared Dowling, which received a 7 at all locations. Thus, the 95B97 Rag1 line has a stronger resistance than Dowling.

### Antixenosis Scale.

9=Equivalent or better when compared to the resistant check—Very few aphids on the plant
7=20-50 aphids on plant, no signs of plant stress
5=50-100 aphids on the plant, moderately susceptible
3=Major damage, including stunting and foliar necrosis
1=Plants are completely covered; severe damage, including severe stunting and necrosis; equivalent or worse when compared to the susceptible check 93B 15.

### Growth Chamber Experiments:

One hundred and eighty F2:3 plants derived from a 95B97×Dowling cross were evaluated for aphid resistance. The isolate used in this study was collected from Lime Springs, Iowa and referred to as biotype X. Seeds were planted two seeds per Conetainer™ (Stuewe and Sons). Seedlings were thinned to one plant per Conetainer™ after emergence. Two bioassays were conducted on each plant. Both bioassays were conducted in a growth chamber with a 16-hour photoperiod.

Bioassay 1 (antixenosis) the aphids are allowed to roam unrestricted on the plants and choose their host. At the V1 stage, the F2:3 segregating plants were infested with seven wingless aphids using a moistened camel hair paintbrush. The soybean genotypes were randomly placed within the Conetainer™ rack. Five replications of each parent were infested and arranged in completely randomized design within a rack placed in a tray filled with water. The trays were watered from the bottom up to avoid disturbing the feeding aphids.

After 7 days, the racks were removed from the growth chamber and rated for aphid infestation. Resistance was evaluated for each plant and rated in the antixenosis scale, where 9=no aphids on the plant, 7=under 10 aphids on the plant, 5=11-50 on plant, and 3=plant is covered. The 95B97 resistant check was rated at a 9 in all experiments and Dowling was rated as a 7 in the choice tests. The aphids preferred Dowling over 95B97 when they had a choice. The aphids placed on 95B97 moved off the plant to feed on other plant hosts. Thus, 95B97 appears to have antixenosis resistance over Dowling.

Bioassay 2 (antibiosis) was conducted on the same plants. Two wingless adults were selected and placed within a double-sided sticky cage on the plant using a moistened camel hair paintbrush. Two cages were placed on each plant. The nonchoice experiment was conducted in the same plant growth chamber with a 16-hour photoperiod. The aphids were allowed to reproduce for 7 days. The plants were removed from the plant and survival, death, and fecundity of the aphids within the cages were recorded. The fecundity was calculated as the mean number of surviving nymphs produced within the cage during the 7-day period for each plant. Plants that had a high rate of nymphal production were classified as susceptible. Plants with some nymphs, but with statistically lower populations compared to the susceptible check were classified as moderately resistant. Plants with no nymph production within the sticky cages and dead or unhealthy in appearance adults were classified as resistant.

The 95B97 donor of Rag1 had no aphid survival in the cages; Dowling had a low level of fecundity within the cages. The progeny from this cross were considered to be resistant when no aphid production occurred and comparable to Dowling when low levels of aphid production occurred within the cage. The aphid scores were rated as:

9=no aphids alive (95B97)

7=a few surviving nymphs within the cage (Dowling)

5=moderately infested (more than 10 within the cage)

3=cages are filled with nymphs and surviving adults

The plants were leaf punched and sampled in collection plates for analysis. The leaf tissue samples were freeze-dried in a lyophilizer and the material was genotyped.

### Example 2

### Genotyping the Mapping Population

DNA was isolated from the collected leaf tissue using standard methods. A total of 235 proprietary SNP markers spaced at ˜10 cM intervals across the genome were used to genotype the entire mapping population. For each marker, the allele calls identical to the maternal parent were assigned “A,” the allele calls identical to the paternal parent were assigned to “B,” the heterozygous alleles were assigned to “H,” and the alleles with “low signal” and “equivocal” assigned to “-” (missing data) in the population. Markers following the expected segregation ratio (p>0.001, chi-square test) were utilized in the initial QTL mapping. The markers with severe segregation distortion (p<0.001) were selected for subsequent mapping analysis.

Map Manager QTX.b20 (Manly et al. (2001) Mammalian Genome 12: 930-932) was used to construct the linkage map and perform the subsequent QTL analysis. The criterion for linkage evaluation was set to p=1e⁻⁵ and Kosambi mapping function was applied to convert the recombination fraction into map distance. The QTL effect was fit into an additive model. A 1000 permutation test was conducted to establish the threshold for statistical significance (LOD ratio statistic—LRS) to declare putative QTL. The mean phenotypic scores from each population were used for the QTL analysis.

This mapping analysis detected three minor QTLs from 95B97, one each on linkage group A2 (LRS=9.5; phenotypic variation explained=7%), linkage group B1 (LRS=14.4; phenotypic variation explained=11%), and linkage group K (LRS=12.4; phenotypic variation explained=9%). Intervals containing these loci are useful, for example, for establishing a favorable marker profile, by which molecular markers would be used to select and introgress this favorable marker profile into soybean varieties carrying the Rag1 gene, or other aphid resistance genes, such as Rag2 or Rag3.

More specifically, this analysis identified five SNP markers that were linked to these QTLs, i.e., markers that were polymorphic between Dowling and 95B97 soybean lines and that were associated with aphid resistance, S03517-1 on LG-A2, S01676-1 on LG-B1, S01675-1 on LG-B1, S00621-1 on LG-K, and 501781-1 on LG-K. Information regarding these markers is provided in Table 1, including map position and primer and probe sequences useful for detecting the markers and the particular SNP allele present. Additionally, the SNP allele present in Dowling and 95B97 for each marker is provided in Table 2.

TABLE 1
SNP markers associated with aphid resistance

| | Linkage | Approximate | Physical | Primer/Probe sequences | | |
|---|---|---|---|---|---|---|
| | Group/ | Relative Map | Position | | Primer | |
| | Chrom. | Position | of SNP | SEQ | or | |
| Marker | No. | (cM) | (bp) | ID: | Probe | Sequence |
| S03517-1 | A2/8 | 115 | 40108281 | 1 | Primer | tgctgtgtactacttcccaagg |
| | | | | 2 | Primer | atggcaaacaagtggggata |
| | | | | 3 | Allelic | ccgagcgttcTaaa |
| | | | | | Probe | |
| | | | | 4 | Allelic | ccgagcgttcCaaa |
| | | | | | Probe | |
| S01629-1 | A2/8 | 105 | 25125032 | 5 | Primer | gatgggttcctttggagtca |
| | | | | 6 | Primer | tcaacaatcatcccctcctc |
| | | | | 7 | Allelic | aagatatgagCtgga |
| | | | | | Probe | |
| | | | | 8 | Allelic | atgagTtggacataag |
| | | | | | Probe | |
| S03253- | B1/11 | 50 | 8971115 | 9 | Primer | aaaacatacccaatcaccatcc |
| 1-A | | | | 10 | Primer | tttcagtggacaacacttctgg |
| | | | | 11 | Allelic | ccaggaaccaAtaca |
| | | | | | Probe | |
| | | | | 12 | Allelic | ccaggaaccaTtacat |
| | | | | | Probe | |
| S01209- | B1/11 | 55 | 9963410 | 13 | Primer | ttcctgaagagcggagacag |
| 1-A | | | | 14 | Primer | gcacggagcttctcataagg |
| | | | | 15 | Allelic | cttccagagcagTgc |
| | | | | | Probe | |
| | | | | 16 | Allelic | ttccagagcagCgc |
| | | | | | Probe | |
| S00737- | B1/11 | 76 | 17389892 | 17 | Primer | gcctgatggtacaccggtcttg |
| 1-A | | | | 18 | Primer | ttgggtctgttgcaccattaagaa |
| | | | | 19 | Allelic | ttcttgagaTatcctcac |
| | | | | | Probe | |
| | | | | 20 | Allelic | ttcttgagaCatcctc |
| | | | | | Probe | |
| S01676-1 | B1/11 | 80 | 18720097 | 21 | Primer | cgctaagcgtgtgctgttat |
| | | | | 22 | Primer | tcacaggaatcaagttagacaggt |
| | | | | 23 | Allelic | ctaagtgccTagtctgt |
| | | | | | Probe | |
| | | | | 24 | Allelic | tcactaagtgccCagtc |
| | | | | | Probe | |
| S01675-1 | B1/11 | 79 | 18387725 | 25 | Primer | tcacaaaaacacacaacatcca |
| | | | | 26 | Primer | accaYatgcatgcaaatcca |
| | | | | 27 | Allelic | tcatacacatagaTacac |
| | | | | | Probe | |
| | | | | 28 | Allelic | tcatacacatagaCacac |
| | | | | | Probe | |
| S04846- | K/9 | 3 | 887780 | 29 | Primer | ggaacaatgtgtcttcaatgct |
| 1-A | | | | 30 | Primer | aagaactagatcatactaactggcatgt |
| | | | | 31 | Allelic | cctaggaatcAgtatgc |
| | | | | | Probe | |
| | | | | 32 | Allelic | cctaggaatcGgtatgc |
| | | | | | Probe | |
| S04864- | K/9 | 6 | 1163103 | 33 | Primer | ctccgttttccccatcttct |
| 1-A | | | | 34 | Primer | agatctctcccgaaacacca |
| | | | | 35 | Allelic | tcgaatcccAgctgt |
| | | | | | Probe | |
| | | | | 36 | Allelic | atcccGgctgtctt |
| | | | | | Probe | |
| S00621-1 | K/9 | 29 | 4700111 | 37 | Primer | tgctttttcctggctttctccc |
| | | | | 38 | Primer | ccaccaggtcgacttggattagttt |
| | | | | 3g | Allelic | caccaTtccaaatgt |
| | | | | | Probe | |
| | | | | 40 | Allelic | accaGtccaaatgt |
| | | | | | Probe | |
| S01781-1 | K19 | 30 | 5021314 | 41 | Primer | aggctaggagcaattggtga |
| | | | | 42 | Primer | caaattggtaccataggcttcttc |
| | | | | 43 | Allelic | actctcaCtggttcc |
| | | | | | Probe | |
| | | | | 44 | Allelic | actctcaGtggttcc |
| | | | | | Probe | |

TABLE 2
Marker alleles in resistant and susceptible soybean lines

| | S03517- | S01629- | S03253- | S01209- | S00737- | S01676- | S01675- | S04846- | S04864- | S00621- | S01781- |
|---|---|---|---|---|---|---|---|---|---|---|---|
| | 1 | 1 | 1-A | 1-A | 1-A | 1 | 1 | 1-A | 1-A | 1 | 1 |
| Susceptible | T | C | A | A | A | G | C | A | A | A | C |
| Allele | | | | | | | | | | | |
| Resistant | C | T | T | G | G | A | T | G | G | C | G |
| Allele | | | | | | | | | | | |

These SNP markers could be useful, for example, for detecting and/or selecting soybean plants with improved aphid resistance. The physical position of each SNP is provided in Table 1, as well. Any marker capable of detecting a polymorphism at one of these physical positions, or a marker linked thereto, could also be useful, for example, for detecting and/or selecting soybean plants with improved aphid resistance. In some examples, the SNP allele present in the 95B97 line could be used as a favorable allele to detect or select plants with improved resistance. In other examples, the SNP allele present in the Dowling line could be used as an unfavorable allele to detect or select plants without improved resistance

These SNP markers could also be used to determine a favorable or unfavorable SNP haplotype and/or a favorable or unfavorable marker profile. In certain examples, a favorable SNP haplotype would include allele “A” for marker S01676-1 and allele “T” for marker S001675-1. In other examples, a favorable SNP haplotype would include allele “C” for marker S00621-1 and allele “G” for marker 501781-1. In other examples, these markers could be used to identify, detect, or select plants displaying a favorable or unfavorable marker profile. In certain examples, a favorable marker profile includes a favorable allele for one or more identified SNP marker located on two or more different linkage groups. Examples of favorable marker profiles are listed in Table 3.

TABLE 3
Favorable aphid resistance marker profiles

| | Link- | Marker | Marker | Marker | Marker | Marker | Marker | Marker | Marker | Marker |
|---|---|---|---|---|---|---|---|---|---|---|
| | age | profile | profile | profile | profile | profile | profile | profile | profile | profile |
| Marker | Group | 1 | 2 | 3 | 4 | 5 | 6 | 7 | 8 | 9 |
| S03517-1 | A2 | C | C | C | C | C | | | | |
| S01676-1 | B1 | A | A | | | | A | A | | |
| S01675-1 | B1 | T | | T | | | | | T | T |
| S00621-1 | K | C | | | C | | C | | C | |
| S01781-1 | K | G | | | | G | | G | | G |
| | | | | | Marker | Marker | Marker | Marker | Marker | Marker |
| | | | | | profile | profile | profile | profile | profile | profile |
| | | | | Marker | 10 | 11 | 12 | 13 | 14 | 15 |
| | | | | S03517-1 | C | C | C | | | C |
| | | | | S01676-1 | A | | | A | A | A |
| | | | | S01675-1 | T | T | T | T | T | |
| | | | | S00621-1 | | C | | C | | C |
| | | | | S01781-1 | | | G | | G | G |

In addition to the SNP markers listed in Table 1, other linked markers could also be useful for detecting and/or selecting soybean plants with improved aphid resistance. Examples of markers linked to the identified SNP markers can be found in the genetic map of FIGS. 1A-1C, 2A-2C, and 3A-3D and the list of closely linked markers in FIGS. 4A-4B. Further, chromosome intervals containing the markers of Table 1 could also be used, such as the interval flanked by and including B132_{—}1 and A065_{—}1 on LG-A2, the interval flanked by and including BLT043_{—}1 and Sat_{—}364 on LG-B1, and the interval flanked by and including BARC-014279-01303 and G214_{—}22 on LG-K.

### Example 3

### Verifying and Identifying Additional Minor QTLs in an F2 Population

### Phenotyping

Three populations of F2 plants were developed for genetic analysis to verify the minor QTLs from the donor variety 95B97. To develop these populations a proprietary Pioneer experimental variety containing the Rag1 donor resistance region derived from 95B97, was crossed with three other Pioneer proprietary soybean lines. The resulting populations were dubbed JB1895, JB1896, and JB1897, respectively. The F2 seeds from the F1 plants were bulked and planted in Conetainer™ units for phenotyping. 360 F2 plants of each population at the V1 stage were inoculated with seven adult females. Populations JB1985 and JB1897 were infested with Biotype 1 aphids, while population JB1896 was infested with Biotype X aphids. The population and its parents were arranged in completely randomized design and placed within Conetainer™ racks. The racks were placed within tents in the growth chamber and the aphids were allowed to move all over the plant or onto neighboring plants. The growth chamber is maintained at 25° C./15° C. day/night temperatures with a 16-hour photoperiod. After 10 days, the plants were evaluated for antixenosis resistance. The susceptible parent rated a 3 in all three bioassays. The resistant donor rated a 9 with no aphids on the plant in all three bioassays. The progeny were rated using the antixenosis scale below.

9=Resistant No aphids on the Plant

8=10 or under on the plant

7=11 to 24 on the plant

6=25 to 50 on the plant

5=(moderately infested) over 50 on the plant

4=Over 100 on the plant but not as covered as 3 rating

3=Plant is completely covered stems and leaves covered (Susceptible)

### Genotyping

For genotyping, the plants from the phenotypic analysis were leaf punched and DNA was isolated from the collected leaf tissue using standard methods. Markers that were determined to be polymorphic between the parents for each population and that flank the QTLs identified in the previous mapping study (on linkage groups A2, B1, and K) were identified. 20, 19, and 18 markers were selected for JB1895, JB1896, and JB1897, respectively.

Preliminary analysis indicated that the populations in JB1895 and JB1897 did not follow the expected 1:2:1 ratio, but followed a 1:2:4 ratio instead. These two populations appear to contain a significant number of progeny resulting from selfing of the susceptible parents. JB1895 contained 148 individuals that match the susceptible parental calls across all 20 markers and JB1897 contained a total of 135 progeny matching the susceptible parental calls across all 18 markers. These progeny were removed from subsequent analysis. JB1895 contained three markers showing segregation distortion (p=0.001), JB1896 had two distorted markers, and JB1897 showed one marker that was distorted. However, all distorted markers were retained in the analysis. One marker returned less than 50% data and another contained no heterozygous calls in JB1896 and both were removed from the analysis. Allele calls for the remaining markers were then converted to the A (maternal), B (paternal), H (heterozygous) convention for QTL analysis. A linkage map was constructed and a subsequent QTL analysis was performed as described for Example 2. Marker regression was performed (p=0.001) across all markers on each population, indicating significant markers on LG-A2 and LG-B1 in JB1895, LG-K in JB1896, and marker S01209-1-A in JB1897. A permutation test was run 1000 times using the free model, establishing the threshold for statistical significance (LOD ratio statistic LRS) to determine putative QTL in JB1895 and JB1896. Interval mapping was then performed using the bootstrap test, free regression model, and the LRS cutoffs determined by the permutation test.

Based on the data from JB1895, a minor QTL was detected on LG-A2 at marker S01629-1-B (LRS=14.0) explaining 6% of the phenotypic variation. Further, a second minor QTL was indicated on LG-B1 between markers S03253-1-A and S01209-1-A (LRS=19.9), explaining 9% of the phenotypic variation. The JB1896 population detected a minor QTL on LG-K between markers S04846-1-A and S04864-1-A (LRS=12.6), explaining 3% of the phenotypic variation. Again, information regarding each of these markers is provided in Table 1 and resistant and susceptible allele calls are provided in Table 2.

Again, these SNP markers could be useful, for example, for detecting and/or selecting soybean plants with improved aphid resistance. The physical position of each SNP is provided in Table 1, as well. Any marker capable of detecting a polymorphism at one of these physical positions, or a marker linked thereto, could also be useful, for example, for detecting and/or selecting soybean plants with improved aphid resistance. Examples of linked markers can be found in FIGS. 1A-1C, 2A-2C, 3A-3D, and 4A-4B. Intervals containing these markers or loci could also be useful. Such intervals could comprise any markers linked to the desired marker or locus as endpoints of the interval, such as the markers listed in FIGS. 4A-4B. In certain embodiments, useful intervals include the chromosome interval flanked by and including BARC-032319-08948 and A065_{—}1 on LG-A2, flanked by and including Sat_{—}250 and Sat_{—}294 on LG-A2, flanked by and including Satt437 and Satt209 on LG-A2, flanked by and including B132_{—}1 and A065_{—}1 on LG-A2, flanked by and including Sat_{—}232 and Sat_{—}294 on LG-A2, flanked by and including Satt333 and Satt209 on LG-A2, flanked by and including BARC-032319-08948 and Sat_{—}138 on LG-A2, flanked by and including Sat_{—}250 and Sat_{—}138 on LG-A2, flanked by and including Satt437 and Satt333 on LG-A2, flanked by and including A847_{—}1 and Sat_{—}364 on LG-B1, flanked by and including Satt197 and Satt430 on LG-B1, flanked by and including A847_{—}1 and BARC-022123-04287 on LG-B1, flanked by and including Satt197 and Satt519 on LG-B1, flanked by and including Satt197 and A520_{—}1 on LG-B1, flanked by and including A847_{—}1 and A520_{—}1 on LG-B1, flanked by and including Satt197 and Sat_{—}149 on LG-B1, flanked by and including Satt197 and Sat_{—}128 on LG-B1, flanked by and including cr122_{—}1 and BARC-022123-04287 on LG-B1, flanked by and including Satt197 and Satt519 on LG-B1, flanked by and including Sat_{—}247 and A520_{—}1 on LG-B1, flanked by and including A006_{—}1 and Sat_{—}364 on LG-B1, flanked by and including Sat_{—}348 and Satt430 on LG-B1, flanked by and including A006_{—}1 and Sat_{—}364 on LG-B1, flanked by and including Sat_{—}348 and Sat_{—}360 on LG-B1, flanked by and including Satt298 and Sat_{—}364 on LG-B1, flanked by and including Satt597 and Satt430 on LG-B1, flanked by and including K401_{—}1 and G214_{—}15 on LG-K, flanked by and including Satt715 and Satt124 on LG-K, flanked by and including K401_{—}1 and BARC-016397-02579 on LG-K, flanked by and including K401_{—}1 and BARC-007972-00189 on LG-K, flanked by and including K401_{—}1 and Sat_{—}087 on LG-K, flanked by and including K401_{—}1 and Satt242 on LG-K, flanked by and including BARC-014279-01303 and G214_{—}15 on LG-K, flanked by and including BARC-039337-07293 and Satt349 on LG-K, flanked by and including BARC-014279-01303 and Sct_{—}196 on LG-K, flanked by and including BARC-039337-07293 and Satt137 on LG-K, flanked by and including A315_{—}1 and G215_{—}15 on LG-K, or flanked by and including Satt055 and Satt349 on LG-K. In other examples, the at least one marker comprises one or more markers within one or more of the genomic DNA regions of SEQ ID NOs: 45, 47, 49, 51, 53, 55, 57, 59, 61, 63, and 65. In other further examples, the one or more marker locus detected comprises one or more markers within one or more of the amplicons of SEQ ID NOs: 46, 48, 50, 52, 54, 56, 58, 60, 62, 64, and 66. In certain examples, the method further comprises detecting one or more marker loci within one or more of the Rag1, Rag2, and Rag3 intervals.

These SNP markers could also be used to determine a favorable or unfavorable SNP haplotype and/or a favorable or unfavorable marker profile. Favorable (resistant) and disfavored (susceptible) allele calls for each of these markers are provided in Table 2. In other examples, these markers could be used to identify, detect, or select plants displaying a favorable or unfavorable marker profile. In certain examples, a favorable marker profile includes a favorable allele for one or more identified SNP marker located on two or more different linkage groups. Examples of favorable marker profiles are listed in Table 3.

What is claimed is:

1. A method of identifying a first soybean plant or germplasm that displays improved resistance to one or more aphid biotypes, the method comprising detecting in the first soybean plant or germplasm at least one allele of one or more marker locus that is associated with the aphid resistance, wherein the one or more marker locus is selected from the group consisting of:
(a) S03517-1 or S01629-1 on linkage group A2;
(b) S03253-1-A, S01209-1-A, S00737-1-A, S01676-1, or S01675-1 on linkage group B1;
(c) S04846-1-A, S04864-1-A, S00621-1, or S01781-1 on linkage group K;
(d) a marker capable of detecting a polymorphism at a physical position selected from the group consisting of 40108281 on LG-A2, 25125032 on LG-A2, 8971115 on LG-B1, 9963410 on LG-B1, 17389892 on LG-B1, 18720097 on LG-B1, 18387725 on LG-B1, 887780 on LG-K, 1163103 on LG-K, 4700111 on LG-K, and 5021314 on LG-K;
(e) a marker locus closely linked to a marker locus of (a), (b), (c), or (d);
(f) a marker locus localizing within a chromosome interval flanked by and including BARC-032319-08948 and A065_{—}1, Satt333 and Satt209, Satt437 and Satt333, or S01629-1 and S03517-1-A on LG-A2;
(g) a marker locus localizing within a chromosome interval flanked by and including A847_{—}1 and Sat_{—}364, A847_{—}1 and BARC-022123-04287, Satt197 and A520_{—}1, A006_{—}1 and Sat_{—}364, Sat_{—}348 and Satt430, S03253-1-A and S01675-1, S003253-1-A and S01209-1-A, or S00737-1-A and S01675-1 on LG-B1;
(h) a marker locus localizing within a chromosome interval flanked by and including K401_{—}1 and G214_{—}15, K401_{—}1 and BARC-016397-02579, BARC-014279-01303 and G214_{—}15, BARC-039337-07293 and Satt349, S04846-1-A and S01781-1-Am S04846-1-A and S04864-1-A, or S00621-1 and S01781-1 on LG-K;
(i) one or more markers within a genomic DNA region selected from the group consisting of SEQ ID NOs: 45, 47, 49, 51, 53, 55, 57, 59, 61, 63, and 65; and
(j) one or more markers within an amplicon selected from the group consisting of SEQ ID NOs: 46, 48, 50, 52, 54, 56, 58, 60, 62, 64, and 66.

2. The method of claim 1, further comprising detecting in the first soybean plant or germplasm at least one of Rag1, Rag2, or Rag3.

3. The method of claim 2, wherein at least two of Rag1, Rag2, or Rag3 are detected.

4. The method of claim 3, wherein Rag1, Rag2, and Rag3 are each detected.

5. The method of claim 1, wherein the one or more marker locus is selected from the group consisting of the marker loci of (a), (b), and (c).

6. The method of claim 1, comprising detecting two or more marker loci of (a)-(j), wherein said two or more marker loci are located on different linkage groups.

7. The method of claim 1, comprising detecting three or more marker loci of (a)-(j) wherein said three or more marker loci are located on different linkage groups.

8. The method of claim 1, wherein the improved resistance is to at least two aphid biotypes.

9. The method of claimed 1, wherein the improved resistance is to three aphid biotypes.

10. The method of claim 1, wherein the detecting comprises amplifying the marker locus or a portion of the marker locus and detecting the resulting amplified marker amplicon.

11. The method of claim 10, wherein the amplifying comprises:
(1) admixing an amplification primer or amplification primer pair with a nucleic acid isolated from the first soybean plant or germplasm, wherein the primer or primer pair is complementary or partially complementary to at least a portion of the marker locus and is capable of initiating DNA polymerization by a DNA polymerase using the soybean nucleic acid as a template; and,
(2) extending the primer or primer pair in a DNA polymerization reaction comprising a DNA polymerase and a template nucleic acid to generate at least one amplicon.

12. The method of claim 11, wherein the nucleic acid is selected from the group consisting of DNA and RNA.

13. The method of claim 10, wherein the at least one allele is an SNP allele, the method comprising detecting the SNP using allele specific hybridization (ASH) analysis or real-time PCR analysis.

14. The method of claim 10, wherein the amplifying comprises employing a polymerase chain reaction (PCR) or ligase chain reaction (LCR).

15. The method of claim 1, wherein the at least one allele is a favorable allele that positively correlates with improved resistance.

16. The method of claim 15, wherein the at least one favorable allele is selected from the group consisting of:
(1) allele C of S03517-1;
(2) allele T of S01629-1;
(3) allele T of S03253-1-A;
(4) allele G of S01209-1-A;
(5) allele G of S00737-1-A;
(6) allele A of S01676-1;
(7) allele T of S01675-1;
(8) allele G of S004846-1-A;
(9) allele G of S04864-1-A;
(10) allele C of S00621-1; and
(11) allele G of S01781-1.

17. The method of claim 15, wherein the at least one favorable allele is selected from the group consisting of:
(1) allele Cat physical position 40108281 on LG-A2;
(2) allele T at physical position 25125032 on LG-A2;
(3) allele T at physical position 8971115 on LG-B1;
(4) allele G at physical position 9963410 on LG-B1;
(5) allele G at physical position 17389892 on LG-B1;
(6) allele A at physical position 18720097 on LG-B1;
(7) allele T at physical position 18387725 on LG-B1;
(8) allele G at physical position 887780 on LG-K;
(9) allele G at physical position 1163103 on LG-K;
(10) allele C at physical position 4700111 on LG-K; and
(11) allele G at physical position 5021314 on LG-K.

18. The method of claim 16, wherein the method comprises detecting all of the favorable alleles (1)-(11).

19. The method of claim 17, wherein the method comprises detecting all of the favorable alleles (1)-(11).

20. The method of claim 1, further comprising selecting the first soybean plant or germplasm, or selecting a progeny of the first soybean plant or germplasm.

21. The method of claim 20, further comprising crossing the selected first soybean plant or germplasm with a second soybean plant or germplasm.

22. The method of claim 21, wherein the second soybean plant or germplasm comprises an exotic soybean strain or an elite soybean strain.

23. A soybean plant or germplasm selected by the method of claim 20.

24. A kit for selecting at least one soybean plant, the kit comprising:
a) primers or probes for detecting one or more marker loci associated with one or more quantitative trait loci associated with improved aphid resistance, wherein the one or more marker loci are selected from the group consisting of:
i) S03517-1 or S01629-1 on linkage group A2;
ii) S03253-1-A, S01209-1-A, S00737-1-A, S01676-1, or S01675-1 on linkage group B1;
iii) S04846-1-A, S04864-1-A, S00621-1, or S01781-1 on linkage group K;
iv) a marker capable of detecting a polymorphism at a physical position selected from the group consisting of 40108281 on LG-A2, 25125032 on LG-A2, 8971115 on LG-B1, 9963410 on LG-B1, 17389892 on LG-B1, 18720097 on LG-B1, 18387725 on LG-B1, 887780 on LG-K, 1163103 on LG-K, 4700111 on LG-K, and 5021314 on LG-K;
v) a marker locus closely linked to a marker locus of (i), (ii), (iii), or (iv);
vi) a marker locus localizing within a chromosome interval flanked by and including BARC-032319-08948 and A065_{—}1, Satt333 and Satt209, Satt437 and Satt333, or S01629-1 and S03517-1-A on LG-A2;
vii) a marker locus localizing within a chromosome interval flanked by and including A847_{—}1 and Sat_{—}364, A847_{—}1 and BARC-022123-04287, Satt197 and A520_{—}1, A006_{—}1 and Sat_{—}364, Sat_{—}348 and Satt430, S03253-1-A and S01675-1, S003253-1-A and S01209-1-A, or S00737-1-A and S01675-1 on LG-B1;
viii) a marker locus localizing within a chromosome interval flanked by and including K401_{—}1 and G214_{—}15, K401_{—}1 and BARC-016397-02579, BARC-014279-01303 and G214_{—}15, BARC-039337-07293 and Satt349, S004846-1-A and S01781-1-Am S004846-1-A and S004864-1-A, or S00621-1 and S01781-1 on LG-K;
ix) one or more markers within a genomic DNA region selected from the group consisting of SEQ ID NOs: 45, 47, 49, 51, 53, 55, 57, 59, 61, 63, and 65; and
x) one or more markers within an amplicon selected from the group consisting of SEQ ID NOs: 46, 48, 50, 52, 54, 56, 58, 60, 62, 64, and 66; and
b) instructions for using the primers or probes for detecting the one or more marker loci and correlating the detected marker loci with predicted tolerance, improved tolerance, susceptibility, or increased susceptibility to aphids.

25. The kit of claim 24, wherein the primers or probes are capable of detecting one or more marker loci selected from the group consisting of S03517-1, S01629-1, S03253-1-A, S01209-1-A, S00737-1-A, S01676-1, S01675-1, S04846-1-A, S04864-1-A, S00621-1, and S01781-1.

26. The kit of claim 25, wherein the primers and probes comprise one or more of SEQ ID NOs: 1-44.

27. An isolated polynucleotide capable of detecting a marker locus selected from the group consisting of S03517-1, S01629-1, S03253-1-A, S01209-1-A, S00737-1-A, S01676-1, S01675-1, S04846-1-A, S04864-1-A, S00621-1, and S01781-1.

28. The isolated polynucleotide of claim 27, wherein the polynucleotide comprises a nucleotide sequence selected from the group consisting of SEQ ID NOs: 1-44, 46, 48, 50, 52, 54, 56, 58, 60, 62, 64, and 66.

29. A seed from the plant of claim 23.

30. A plant cell derived from the plant of claim 23.

31. A soybean plant or germplasm comprising at least one allele of one or more marker locus that is associated with the aphid resistance, wherein the one or more marker locus is selected from the group consisting of:
(a) S03517-1 or S01629-1 on linkage group A2;
(b) S03253-1-A, S01209-1-A, S00737-1-A, S01676-1, or S01675-1 on linkage group B1;
(c) S04846-1-A, 504864-1-A, S00621-1, or S01781-1 on linkage group K;
(d) a marker capable of detecting a polymorphism at a physical position selected from the group consisting of 40108281 on LG-A2, 25125032 on LG-A2, 8971115 on LG-B1, 9963410 on LG-B1, 17389892 on LG-B1, 18720097 on LG-B1, 18387725 on LG-B1, 887780 on LG-K, 1163103 on LG-K, 4700111 on LG-K, and 5021314 on LG-K;
(e) a marker locus closely linked to a marker locus of (a), (b), (c), or (d);
(f) a marker locus localizing within a chromosome interval flanked by and including BARC-032319-08948 and A065_{—}1, Satt333 and Satt209, Satt437 and Satt333, or S01629-1 and S03517-1-A on LG-A2;
(g) a marker locus localizing within a chromosome interval flanked by and including A847_{—}1 and Sat_{—}364, A847_{—}1 and BARC-022123-04287, Satt197 and A520_{—}1, A006_{—}1 and Sat_{—}364, Sat_{—}348 and Satt430, S03253-1-A and S01675-1, S03253-1-A and S01209-1-A, or S00737-1-A and S01675-1 on LG-B1;
(h) a marker locus localizing within a chromosome interval flanked by and including K401_{—}1 and G214_{—}15, K401_{—}1 and BARC-016397-02579, BARC-014279-01303 and G214_{—}15, BARC-039337-07293 and Satt349, S04846-1-A and S01781-1-Am S04846-1-A and S04864-1-A, or S00621-1 and S01781-1 on LG-K;
(i) one or more markers within a genomic DNA region selected from the group consisting of SEQ ID NOs: 45, 47, 49, 51, 53, 55, 57, 59, 61, 63, and 65; and
(j) one or more markers within an amplicon selected from the group consisting of SEQ ID NOs: 46, 48, 50, 52, 54, 56, 58, 60, 62, 64, and 66;
wherein said soybean plant or germplasm further comprises at least one of Rag1, Rag2, or Rag3.

32. The soybean plant or germplasm of claim 31, wherein said soybean plant or germplasm comprises at least two of Rag1, Rag2, or Rag3 are detected.

33. The soybean plant or germplasm of claim 31, wherein said soybean plant or germplasm comprises Rag1, Rag2, and Rag3.

34. The soybean plant or germplasm of claim 31, wherein the one or more marker locus is selected from the group consisting of the marker loci of (a), (b), and (c).

35. The soybean plant or germplasm of claim 31, wherein said soybean plant or germplasm comprises two or more marker loci of (a)-(j), wherein said two or more marker loci are located on different linkage groups.

36. The soybean plant or germplasm of claim 31, wherein said soybean plant or germplasm comprises three or more marker loci of (a)-(j) wherein said three or more marker loci are located on different linkage groups.

37. A seed from the soybean plant or germplasm of claim 31.

38. A plant cell derived from the soybean plant or germplasm of claim 31.
